(19) United States
(12) Patent Application Publication
(10) Pub. No.: US 2025/0277191 A1
(43) Pub. Date: Sep. 4, 2025

(54) METHODS AND COMPOSITIONS FOR CRYOPRESERVING SUBPOPULATIONS OF LYMPHOCYTES

(71) Applicant: Ossium Health, Inc., San Francisco, CA (US)

(72) Inventors: Erik J. Woods, Carmel, IN (US); Jianguang Du, Carmel, IN (US); Brian H. Johnstone, Fishers, IN (US)

(21) Appl. No.: 19/015,356

(22) Filed: Jan. 9, 2025

Related U.S. Application Data
(63) Continuation of application No. PCT/US2023/069991, filed on Jul. 11, 2023.
(60) Provisional application No. 63/388,115, filed on Jul. 11, 2022.

(51) Int. Cl. C12N 5/00; C12N 5/078

(52) U.S. Cl. CPC C12N 5/562; C12N 5/0634; C12N 5/525

(57) ABSTRACT

Provided are methods and compositions for cryopreserving subpopulations of lymphocytes. In one aspect, provided herein is a method of generating a sub-population of lymphocytes from a biological sample, the method comprising cryopreserving said biological sample in a cryopreserving solvent comprising less than 2M molar concentration of an active cryopreserving moiety. In another aspect, provided herein is a cellular composition comprising an enhanced sub-population of lymphocytes, an enhanced second sub-population of lymphocytes, and a depleted third subpopulation of lymphocytes, wherein said cellular composition further comprises a cryopreserving solvent comprising less than 2M molar concentration of an active cryopreserving moiety. In another aspect, provided herein is a method for preparing an allogenic composition from a biological sample, wherein said allogenic composition comprises an enhanced sub-population of lymphocytes, the method comprising cryopreserving said biological sample in a cryopreserving solvent comprising less than 2M molar concentration of an active cryopreserving moiety.

### CROSS-REFERENCE TO RELATED APPLICATIONS

This application is a continuation application of PCT Application No. PCT/US2023/069991, filed Jul. 11, 2023, which claims priority to U.S. Provisional Application No. 63/388,115 filed Jul. 11, 2022. The entire contents of each of the aforementioned patent applications is incorporated herein by reference.

### BACKGROUND

Hematopoietic cell transplantation (HCT) and bone marrow transplantation (BMT) are potentially curative treatments for many patients with malignant and non-malignant hematological disorders. Successful treatment to establish remission critically depends on intrinsic graft-versus-leukemia (GVL) effects mediated by lymphocytes within the donor HCT/BMT graft.

Unfortunately, activity against recipient tissues is not confined to malignant cells, often resulting in injury to normal tissues, termed graft-versus-host-disease (GVHD). Both GVL and GVHD process involve donor lymphocytes responding to genetic differences to recipient tissues. Although the two processes are related, multiple lines of evidence support the nonoverlapping roles of various discrete lymphocyte subsets, which has led to intense research efforts directed at separating the two activities with the goal of selectively enhancing GVL effects while abrogating or at least not enhancing inherent GVHD.

Studies with T cell depleted HLA-mismatched grafts demonstrated clearance of leukemic cells through the reactivity of donor natural killer (NK) cells against recipient hematopoietic cells without targeting non-hematopoietic cells involved in GVHD. Evidence is also accumulating for a primary role of non-conventional T cell receptor (TCR) γ δ T cells, as opposed to conventional TCRγ δ T cells, as mediators of allo-HCT GVL effects with a possible protective role against GVHD. On the other hand, unless the donor has been sensitized through blood transfusion or pregnancy, alloimmune lymphocytes which contribute to GVHD naturally reside within the naïve T cell subset. Thus, current evidence suggests that GVL and GVHD functions of HCT/BMT are associated with distinct lymphocyte populations within the graft which are potentially separable.

Present strategies to selectively modify lymphocyte subpopulations within HCT/BMT grafts rely on immunomagnetic bead-based cell depletion. This approach has been used to separately remove TCR αβ T cells (in combination with CD19+ B cells) or CD45RA+ naïve T cells. An alternative strategy is to purify and expand lymphocyte subsets, with or without genetic manipulation, for subsequent adoptive transfer. Treatment with ex vivo expanded cytotoxic lymphocyte subsets builds on unmanipulated donor lymphocyte infusion (DLI) which was been used for decades. Regulatory T cells (Tregs) are another T cell subset that normally serve to maintain immunological self-tolerance and under pathological conditions modulate alloreactivity of effector memory T (Tem) cells and have been shown to reduce GVHD following adoptive transfer.

### SUMMARY

In certain aspects, described herein is a method of generating a sub-population of lymphocytes from a biological sample, the method comprising cryopreserving said biological sample in a cryopreserving solvent comprising less than 2M molar concentration of an active cryopreserving moiety. In some embodiments, the method further comprises generating a second sub-population of lymphocytes from said biological sample. In some embodiments, the method further comprises depleting a third sub-population of lymphocytes from said biological sample. In some embodiments, said sub-population of lymphocytes comprises gd T cells. In some embodiments, said second sub-population of lymphocytes comprises CD8+ naïve T cells. In some embodiments, said third sub-population of lymphocytes comprises CD4+ naïve T cells. In some embodiments, said biological sample is derived from a donor subject. In some embodiments, said biological sample comprises blood. In some embodiments, said biological sample comprises bone marrow, peripheral blood, cord blood, or any combination thereof. In some embodiments, said active cryopreserving moiety comprises an amphiphilic compound. In some embodiments, said active cryopreserving moiety comprises a polar aprotic compound. In some embodiments, said active cryopreserving moiety comprises a zwitterionic compound. In some embodiments, said active cryopreserving moiety comprises dimethyl sulfoxide (DMSO); 1, 2 propane diol; propylene glycol; ethylene glycol; glycerol; foramamide; ethanediol or butane 2, 3 diol. In some embodiments, said cryopreserving solvent comprises less than 1.7M, less than 1.4M, less than 1.1M, less than 0.8M, less than 0.5M, or less than 0.2M molar concentration of said active cryopreserving moiety. In some embodiments, said sub-population of lymphocytes comprises greater than 0.2% of said biological sample. In some embodiments, said second sub-population of lymphocytes comprises greater than 20% of said biological sample. In some embodiments, said third sub-population of lymphocytes comprises less than 60% of said biological sample. In some embodiments, the method further comprises generated a population of natural killer (NK) cells. In some embodiments, said population of NK cells comprises greater than 0.1% of said biological sample. In some embodiments, said cryopreserving solvent comprises less than 10%, less than 9%, less than 8%, less than 7%, less than 6%, or less than 5% of said active cryopreserving moiety.

In certain aspects, described herein is a cellular composition comprising an enhanced sub-population of lymphocytes, an enhanced second sub-population of lymphocytes, and a depleted third subpopulation of lymphocytes, wherein said cellular composition further comprises a cryopreserving solvent comprising less than 2M molar concentration of an active cryopreserving moiety. In some embodiments, said sub-population of lymphocytes comprises gd T cells. In some embodiments, said second sub-population of lymphocytes comprises CD8+ naïve T cells. In some embodiments, said third sub-population of lymphocytes comprises CD4+ naïve T cells. In some embodiments, said biological sample is derived from a donor subject. In some embodiments, said biological sample comprises blood. In some embodiments, said biological sample comprises bone marrow, peripheral blood, cord blood, or any combination thereof. In some embodiments, said active cryopreserving moiety comprises an amphiphilic compound. In some embodiments, said active cryopreserving moiety comprises a polar aprotic compound. In some embodiments, said active cryopreserving moiety comprises a zwitterionic compound. In some embodiments, said active cryopreserving moiety comprises dimethyl sulfoxide (DMSO); 1, 2 propane diol; propylene glycol; ethylene glycol; glycerol; foramamide; ethanediol or butane 2, 3 diol. In some embodiments, said cryopreserving solvent comprises less than 1.7M, less than 1.4M, less than 1.1M, less than 0.8M, less than 0.5M, or less than 0.2M molar concentration of said active cryopreserving moiety. In some embodiments, said sub-population of lymphocytes comprises greater than 0.2% of said biological sample. In some embodiments, said second sub-population of lymphocytes comprises greater than 20% of said biological sample. In some embodiments, said third sub-population of lymphocytes comprises less than 60% of said biological sample. In some embodiments, the method further comprises generated a population of natural killer (NK) cells. In some embodiments, said population of NK cells comprises greater than 0.1% of said biological sample. In some embodiments, the cellular composition is allogenic. In some embodiments, said cryopreserving solvent comprises less than 10%, less than 9%, less than 8%, less than 7%, less than 6%, or less than 5% of said active cryopreserving moiety.

In certain aspects, described herein is a method for preparing an allogenic composition from a biological sample, wherein said allogenic composition comprises an enhanced sub-population of lymphocytes, the method comprising cryopreserving said biological sample in a cryopreserving solvent comprising less than 2M molar concentration of an active cryopreserving moiety. In some embodiments, said allogenic composition further comprises an enhanced second sub-population of lymphocytes from said biological sample. In some embodiments, said allogenic composition further comprises a depleted third sub-population of lymphocytes from said biological sample. In some embodiments, said sub-population of lymphocytes comprises gd T cells. In some embodiments, said second sub-population of lymphocytes comprises CD8+ naïve T cells. In some embodiments, said third sub-population of lymphocytes comprises CD4+ naïve T cells. In some embodiments, said biological sample is derived from a donor subject. In some embodiments, said biological sample comprises blood. In some embodiments, said biological sample comprises bone marrow, peripheral blood, cord blood, or any combination thereof. In some embodiments, said active cryopreserving moiety comprises an amphiphilic compound. In some embodiments, said active cryopreserving moiety comprises a polar aprotic compound. In some embodiments, said active cryopreserving moiety comprises a zwitterionic compound. In some embodiments, said active cryopreserving moiety comprises dimethyl sulfoxide (DMSO); 1, 2 propane diol; propylene glycol; ethylene glycol; glycerol; foramamide; ethanediol or butane 2, 3 diol. In some embodiments, said cryopreserving solvent comprises less than 1.7M, less than 1.4M, less than 1.1M, less than 0.8M, less than 0.5M, or less than 0.2M molar concentration of said active cryopreserving moiety. In some embodiments, said sub-population of lymphocytes comprises greater than 0.2% of said allogenic composition. In some embodiments, said second sub-population of lymphocytes comprises greater than 20% of said allogenic composition. In some embodiments, said third sub-population of lymphocytes comprises less than 60% of said allogenic composition. In some embodiments, said allogenic composition further comprises an enhanced population of natural killer (NK) cells. In some embodiments, said enhanced population of NK cells comprises greater than 0.1% of said allogenic composition. In some embodiments, said cryopreserving solvent comprises less than 10%, less than 9%, less than 8%, less than 7%, less than 6%, or less than 5% of said active cryopreserving moiety.

In certain aspects, described herein is a method of treating an auto-immune disease in a subject in need thereof, the method comprising: cryopreserving a biological sample in a cryopreserving solvent comprising less than 2M molar concentration of an active cryopreserving moiety to generate an allogenic composition, wherein said allogenic composition comprises an enhanced sub-population of lymphocytes; and administering said allogenic composition to said subject. In some embodiments, said allogenic composition further comprises an enhanced second sub-population of lymphocytes from said biological sample. In some embodiments, said allogenic composition further comprises a depleted third sub-population of lymphocytes from said biological sample. In some embodiments, said sub-population of lymphocytes comprises gd T cells. In some embodiments, said second sub-population of lymphocytes comprises CD8+ naïve T cells. In some embodiments, said third sub-population of lymphocytes comprises CD4+ naïve T cells. In some embodiments, said biological sample is derived from a donor subject. In some embodiments, said biological sample comprises blood. In some embodiments, said biological sample comprises bone marrow, peripheral blood, cord blood, or any combination thereof. In some embodiments, said active cryopreserving moiety comprises an amphiphilic compound. In some embodiments, said active cryopreserving moiety comprises a polar aprotic compound. In some embodiments, said active cryopreserving moiety comprises a zwitterionic compound. In some embodiments, said active cryopreserving moiety comprises dimethyl sulfoxide (DMSO); 1, 2 propane diol; propylene glycol; ethylene glycol; glycerol; foramamide; ethanediol or butane 2, 3 diol. In some embodiments, said cryopreserving solvent comprises less than 1.7M, less than 1.4M, less than 1.1M, less than 0.8M, less than 0.5M, or less than 0.2M molar concentration of said active cryopreserving moiety. In some embodiments, said sub-population of lymphocytes comprises greater than 0.2% of said allogenic composition. In some embodiments, said second sub-population of lymphocytes comprises greater than 20% of said allogenic composition. In some embodiments, said third sub-population of lymphocytes comprises less than 60% of said allogenic composition. In some embodiments, said allogenic composition further comprises an enhanced population of natural killer (NK) cells. In some embodiments, said enhanced population of NK cells comprises greater than 0.1% of said allogenic composition. In some embodiments, said auto immune disease is graft-versus-host disease. In some embodiments, said cryopreserving solvent comprises less than 10%, less than 9%, less than 8%, less than 7%, less than 6%, or less than 5% of said active cryopreserving moiety.

In certain aspects, described herein is a method of preventing occurrence of an auto-immune disease in a subject receiving an organ transplant, the method comprising: cryopreserving a biological sample derived from a donor subject in a cryopreserving solvent comprising less than 2M molar concentration of an active cryopreserving moiety to generate an allogenic composition, wherein said allogenic composition comprises an enhanced sub-population of lymphocytes; and administering said allogenic composition to said subject receiving said organ transplant. In some embodiments, said allogenic composition further comprises an enhanced second sub-population of lymphocytes from said biological sample. In some embodiments, said allogenic composition further comprises a depleted third sub-population of lymphocytes from said biological sample. In some embodiments, said sub-population of lymphocytes comprises gd T cells. In some embodiments, said second sub-population of lymphocytes comprises CD8+ naïve T cells. In some embodiments, said third sub-population of lymphocytes comprises CD4+ naïve T cells. In some embodiments, said biological sample comprises blood. In some embodiments, said biological sample comprises bone marrow, peripheral blood, cord blood, or any combination thereof. In some embodiments, said active cryopreserving moiety comprises an amphiphilic compound. In some embodiments, said active cryopreserving moiety comprises a polar aprotic compound. In some embodiments, said active cryopreserving moiety comprises a zwitterionic compound. In some embodiments, said active cryopreserving moiety comprises dimethyl sulfoxide (DMSO); 1, 2 propane diol; propylene glycol; ethylene glycol; glycerol; foramamide; ethanediol or butane 2, 3 diol. In some embodiments, said cryopreserving solvent comprises less than 1.7M, less than 1.4M, less than 1.1M, less than 0.8M, less than 0.5M, or less than 0.2M molar concentration of said active cryopreserving moiety. In some embodiments, said sub-population of lymphocytes comprises greater than 0.2% of said allogenic composition. In some embodiments, said second sub-population of lymphocytes comprises greater than 20% of said allogenic composition. In some embodiments, said third sub-population of lymphocytes comprises less than 60% of said allogenic composition. In some embodiments, said allogenic composition further comprises an enhanced population of natural killer (NK) cells. In some embodiments, said enhanced population of NK cells comprises greater than 0.1% of said allogenic composition. In some embodiments, said auto immune disease is graft-versus-host disease. In some embodiments, said cryopreserving solvent comprises less than 10%, less than 9%, less than 8%, less than 7%, less than 6%, or less than 5% of said active cryopreserving moiety.

In certain aspects, described herein is a kit for preparing an allogenic composition to prevent or treat an auto immune disease in a subject in need thereof, wherein said allogenic composition comprises an enhanced sub-population of lymphocytes, the kit comprising a cryopreserving solvent comprising less than 2M molar concentration of an active cryopreserving moiety. In some embodiments, said allogenic composition further comprises an enhanced second sub-population of lymphocytes from said biological sample. In some embodiments, said allogenic composition further comprises a depleted third sub-population of lymphocytes from said biological sample. In some embodiments, said sub-population of lymphocytes comprises gd T cells. In some embodiments, said second sub-population of lymphocytes comprises CD8+ naïve T cells. In some embodiments, said third sub-population of lymphocytes comprises CD4+ naïve T cells. In some embodiments, said biological sample comprises blood. In some embodiments, said biological sample comprises bone marrow, peripheral blood, cord blood, or any combination thereof. In some embodiments, said active cryopreserving moiety comprises an amphiphilic compound. In some embodiments, said active cryopreserving moiety comprises a polar aprotic compound. In some embodiments, said active cryopreserving moiety comprises a zwitterionic compound. In some embodiments, said active cryopreserving moiety comprises dimethyl sulfoxide (DMSO); 1, 2 propane diol; propylene glycol; ethylene glycol; glycerol; foramamide; ethanediol or butane 2, 3 diol; hydroxyethyl starch (HES); dextran; sucrose; trehalose; lactose; raffinose; ribotol; mannitol; and polyvinylpyrrolidone (PVP). In some embodiments, said cryopreserving solvent comprises less than 1.7M, less than 1.4M, less than 1.1M, less than 0.8M, less than 0.5M, or less than 0.2M molar concentration of said active cryopreserving moiety. In some embodiments, said sub-population of lymphocytes comprises greater than 0.2% of said allogenic composition. In some embodiments, said second sub-population of lymphocytes comprises greater than 20% of said allogenic composition. In some embodiments, said third sub-population of lymphocytes comprises less than 60% of said allogenic composition. In some embodiments, said allogenic composition further comprises an enhanced population of natural killer (NK) cells. In some embodiments, said enhanced population of NK cells comprises greater than 0.1% of said allogenic composition. In some embodiments, said auto immune disease is graft-versus-host disease. In some embodiments, said cryopreserving solvent comprises less than 10%, less than 9%, less than 8%, less than 7%, less than 6%, or less than 5% of said active cryopreserving moiety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A to FIG. 1F depicts different populations of bone marrow cells present in Hematopoietic cells and bone marrow were measured by flow cytometry before (“fresh BM”) and after (“cryo BM”) cryopreservation in 5% DMSO. (FIG. 1A) CD45+CD34+ HSPC; (FIG. 1B); total CD+ T cells; (FIG. 1C) CD3+CD4−CD8−γ 8 TCR+δ & δ cells; (FIG. 1D) CD4+CD45RA+CCR7+ naïve T cells; (FIG. 1E) CD8+CD45RA+CCR7+ naïve T cells; and (FIG. 1F) CD3−CD19−CD56+ NK cells. Percentages of each population were calculated from either total viable cells (“of viable TNC”) or specific viable T cell populations (“of viable CD4+ T cells” or “of viable CD8+ T cells”). **, P<0.01; ***, P<0.001.

FIG. 2A to FIG. 2F depicts different populations of bone marrow cells present in Hematopoietic cells and bone marrow cryopreserved in either 2.5, 5 or 10% DMSO were determined by flow cytometry. (FIG. 2A) CD45^{LO}CD34+ HSPC; (FIG. 2B) CD3−CD19−CD56+ NK cells; (FIG. 2C) CD3+CD4−CD8− γ δ TCR+δ & δ cells; (FIG. 2D) CD4+CD45RA+CCR7+ naïve T cells; (FIG. 2E) CD8+CD45RA+CCR7+ naïve T cells; and (FIG. 2F) CD3-CD19-CD56+NK cells. Percentages of each population calculated from either total viable cells (“of viable TNC”) or specific viable T cell populations (“of viable CD4+ T cells” or “of viable CD8* T cells”). NS, not significant (P>0.05); *, P<0.05; **, P<0.01; ***, P<0.001.

FIG. 3 is a flowchart of one method according to the present disclosure.

FIG. 4 is a perspective view of a cooling box according to an aspect of the present disclosure.

FIG. 5 illustrates HPC, Marrow experimental trials cassette location in shelf one of the −86° C. Eppendorf Cryocube Model F740hi.

FIG. 6 illustrates an exemplary alternative arrangement of the cassette location in shelf one of the −86° C. Eppendorf Cryocube Model F740hi.

### DETAILED DESCRIPTION

Provided herein are methods, compositions, and kits for generating a sub-population of lymphocytes from a biological sample, the method comprising cryopreserving said biological sample in a cryopreserving solvent. These embodiments provide useful applications for generating desirable cell populations, for example, gd T cells.

Applications of the embodiments disclosed herein efficiently produce desired cell populations. For example, applications of the methods described herein do not require additional cell culturing over the course of multiple days to produce and/or purify a desired cell population, like gd T cells. Instead, the cryopreservation techniques described herein provide for specific conditions, for example, providing a biological sample (e.g. human bone marrow) in a cryopreserving solvent comprising less than 2M molar concentration of an active cryopreserving moiety (e.g. a 2.5% dimethyl sulfoxide [DMSO] solution), to specifically select for a desired sub-population of cells, like gd T cells.

Generating and/or purifying sub-populations of cells from a biological sample by means of specific cryopreservation techniques can be applied to generating therapeutic products. For example, gd T cells are useful for treating certain diseases characterized by abnormally active immune systems, like autoimmune diseases (e.g. graft-versus-host-disease or GVHD). In some embodiments, of the disclosure, the cryopreservation techniques can be used to generate gd T cells that can be used for treating autoimmune diseases like GVHD.

While autoimmune diseases are common, autoimmune diseases like GVHD are particularly common in subjects that have received a biological transplant (e.g. organ transplant or solid organ transplant), as the host-subject's immune system recognizes the grafted transplant. The embodiments disclosed herein are particularly useful for producing and/or purifying sub-populations of cells that are particularly useful in application with an organ transplant.

### Compositions

In some embodiments, described herein is a cellular composition derived from a biological sample. In some embodiments, the composition comprises an enhanced sub-population of lymphocytes, an enhanced second sub-population of lymphocytes, and a depleted third subpopulation of lymphocytes. In some embodiments, the cellular composition further comprises a cryopreserving solvent.

### Biological Samples

The methods described herein comprise generating a sub-population of lymphocytes from a biological sample. In some embodiments, the biological sample comprises bone marrow, peripheral blood, cord blood, or any combination thereof. In some embodiments, the biological sample is blood. In some embodiments, the biological sample is bone marrow.

In some embodiments, the biological sample is derived from a donor. In some instances, the bone marrow can be obtained from a deceased donor. In some cases, the bone marrow can be obtained from a sample (e.g. bone or VB) that was previously chilled. In some cases, the bone marrow can be obtained from a sample (e.g. bone or VB) that was previously chilled but not frozen. In some cases, the bone marrow can be obtained from a sample (e.g. bone or VB) that is thawed. In some cases, the bone marrow can be processed for obtaining bone marrow cells. In some embodiments, the bone marrow cells can be hematopoietic stem cells (HSCs). In some embodiments, the bone marrow cells can be mesenchymal stem cells (MSCs).

### Lymphocytes and Subpopulations

In some embodiments, the bone marrow cells comprise a subpopulation of lymphocytes. In some embodiments, the sub-population of lymphocytes comprises B cells or T cells. In some embodiments, the sub-population of lymphocytes comprises γδ T cells, CD8+ naïve T cells, CD4+ naïve T cells, or Natural Killer (NK) cells.

In some embodiments, the sub-population of lymphocytes comprises greater than 0.1%, 0.2%, 0.3%, 04%, 0.5%, 0.6%, 0.7%, 0.8%, 0.9%, or 1% of said biological sample. In some embodiments, the sub-population of lymphocytes comprises greater than 0.1% of said biological sample. In some embodiments, the sub-population of lymphocytes comprises greater than 0.2% of said biological sample. In some embodiments, the sub-population of lymphocytes comprises greater than 0.3% of said biological sample. In some embodiments, the sub-population of lymphocytes comprises greater than 0.4% of said biological sample. In some embodiments, the sub-population of lymphocytes comprises greater than 0.5% of said biological sample. In some embodiments, the sub-population of lymphocytes comprises greater than 0.6% of said biological sample. In some embodiments, the sub-population of lymphocytes comprises greater than 0.7% of said biological sample. In some embodiments, the sub-population of lymphocytes comprises greater than 0.8% of said biological sample. In some embodiments, the sub-population of lymphocytes comprises greater than 0.9% of said biological sample. In some embodiments, the sub-population of lymphocytes comprises greater than 1% of said biological sample.

In some embodiments, the sub-population of lymphocytes comprises a second sub-population. In some embodiments, the second sub-population of lymphocytes comprises gd T cells. In some embodiments, the second sub-population of lymphocytes comprises CD8+ naïve T cells. In some embodiments, said second sub-population of lymphocytes comprises greater than 10%, 20%, 30%, 40%, 50%, 60% or more of said biological sample. In some embodiments, said second sub-population of lymphocytes comprises greater than 10% or more of said biological sample. In some embodiments, said second sub-population of lymphocytes comprises greater than 20% or more of said biological sample. In some embodiments, said second sub-population of lymphocytes comprises greater than 30% or more of said biological sample. In some embodiments, said second sub-population of lymphocytes comprises greater than 40% or more of said biological sample. In some embodiments, said second sub-population of lymphocytes comprises greater than 50% or more of said biological sample. In some embodiments, said second sub-population of lymphocytes comprises greater than 60% or more of said biological sample.

In some embodiments, the sub-population of lymphocytes comprises a third sub-population. In some embodiments, the third sub-population comprises CD4+ naïve T cells. In some embodiments, said third sub-population of lymphocytes comprises less than 30%, 40%, 50%, 60%, 70%, 80% or 90% of said biological sample. In some embodiments, said third population of lymphocytes comprises less than 30% of said biological sample. In some embodiments, said third population of lymphocytes comprises less than 40% of said biological sample. In some embodiments, said third population of lymphocytes comprises less than 50% of said biological sample. In some embodiments, said third population of lymphocytes comprises less than 60% of said biological sample. In some embodiments, said third population of lymphocytes comprises less than 70% of said biological sample. In some embodiments, said third population of lymphocytes comprises less than 80% of said biological sample. In some embodiments, said third population of lymphocytes comprises less than 90% of said biological sample.

In some embodiments, the bone marrow cells comprises a population of natural killer (NK) cells. In some embodiments, the population of NK cells comprises greater than 0.01%, 0.02%, 0.03%, 0.04%, 0.05%, 0.06%, 0.07%, 0.08%, 0.09%, 0.1%, 0.2%, 0.3%, 04%, 0.5%, 0.6%, 0.7%, 0.8%, 0.9%, or 1% of said biological sample. In some embodiments, the population of NK cells comprises greater than 0.01% of said biological sample. In some embodiments, the population of NK cells comprises greater than 0.02% of said biological sample. In some embodiments, the population of NK cells comprises greater than 0.03% of said biological sample. In some embodiments, the population of NK cells comprises greater than 0.04% of said biological sample. In some embodiments, the population of NK cells comprises greater than 0.05% of said biological sample. In some embodiments, the population of NK cells comprises greater than 0.06% of said biological sample. In some embodiments, the population of NK cells comprises greater than 0.07% of said biological sample. In some embodiments, the population of NK cells comprises greater than 0.08% of said biological sample. In some embodiments, the population of NK cells comprises greater than 0.09% of said biological sample. In some embodiments, the population of NK cells comprises greater than 0.1% of said biological sample. In some embodiments, the population of NK cells comprises greater than 0.2% of said biological sample. In some embodiments, the population of NK cells comprises greater than 0.3% of said biological sample. In some embodiments, the population of NK cells comprises greater than 0.4% of said biological sample. In some embodiments, the population of NK cells comprises greater than 0.5% of said biological sample. In some embodiments, the population of NK cells comprises greater than 0.6% of said biological sample. In some embodiments, the population of NK cells comprises greater than 0.7% of said biological sample. In some embodiments, the population of NK cells comprises greater than 0.8% of said biological sample. In some embodiments, the population of NK cells comprises greater than 0.9% of said biological sample. In some embodiments, the population of NK cells comprises greater than 1% of said biological sample.

### Cryopreserving Moieties

In some embodiments, the active cryopreserving moiety comprises a polar aprotic compound or a zwitterionic compound. In some embodiments, the active cryopreserving moiety comprises a polar aprotic compound. In some embodiments, said active cryopreserving moiety comprises a zwitterionic compound. In some embodiments, the active cryopreserving moiety comprises dimethyl sulfoxide (DMSO); 1, 2 propane diol; propylene glycol; ethylene glycol; glycerol; foramamide; ethanediol or butane 2, 3 diol.

In some embodiments, the cryopreserving solvent comprises less than 1.7M, less than 1.4M, less than 1.1M, less than 0.8M, less than 0.5M, or less than 0.2M molar concentration of said active cryopreserving moiety. In some embodiments, the cryopreserving solvent comprises less than 1.7M concentration of said active cryopreserving solvent. In some embodiments, the cryopreserving solvent comprises less than 1.4M concentration of said active cryopreserving solvent. In some embodiments, the cryopreserving solvent comprises less than 1.1M concentration of said active cryopreserving solvent. In some embodiments, the cryopreserving solvent comprises less than 0.8M concentration of said active cryopreserving solvent. In some embodiments, the cryopreserving solvent comprises less than 0.5M concentration of said active cryopreserving solvent. In some embodiments, the cryopreserving solvent comprises less than 0.2M concentration of said active cryopreserving solvent.

In some embodiments, the cryopreserving solvent comprises less than 10%, less than 9%, less than 8%, less than 7%, less than 6%, or less than 5% of said active cryopreserving moiety. In some embodiments, the cryopreserving solvent comprises less than 10% of said active cryopreserving solvent. n some embodiments, the cryopreserving solvent comprises less than 9% of said active cryopreserving solvent. n some embodiments, the cryopreserving solvent comprises less than 8% of said active cryopreserving solvent. n some embodiments, the cryopreserving solvent comprises less than 7% of said active cryopreserving solvent. n some embodiments, the cryopreserving solvent comprises less than 6% of said active cryopreserving solvent. In some embodiments, the cryopreserving solvent comprises less than 5% of said active cryopreserving solvent.

### Methods

Described herein in certain aspects, is a method of generating a sub-population of lymphocytes from a biological sample, the method comprising cryopreserving the biological sample in a cryopreserving solvent described herein. In some embodiments, the sub-population of lymphocytes is administered to a subject to treat an autoimmune disease. In some embodiments, the sub-population of lymphocytes is administered to a subject receiving an organ transplant to prevent occurrence of an autoimmune disease.

In some embodiments, the methods comprise generating a second sub-population of lymphocytes from the biological sample. In some embodiments, the sub-population of lymphocytes comprises a second sub-population. In some embodiments, the second sub-population of lymphocytes comprises gd T cells. In some embodiments, the second sub-population of lymphocytes comprises CD8+ naïve T cells. In some embodiments, said second sub-population of lymphocytes comprises greater than 10%, 20%, 30%, 40%, 50%, 60% or more of said biological sample.

In some embodiments, the method further comprises depleting a third sub-population of lymphocytes from the biological sample. In some embodiments, the third sub-population comprises CD4+ naïve T cells. In some embodiments, the third sub-population of lymphocytes comprises less than 30%, 40%, 50%, 60%, 70%, 80% or 90% of said biological sample.

In some embodiments, the sub-population of lymphocytes described herein is administered to a subject. In some embodiments, the subject has an autoimmune disease. In some embodiments, the autoimmune disease is selected from the group consisting of Lupus, liver cirrhosis, osteon arthritis, and rheumatoid arthritis. In some embodiments, the sub-population of lymphocytes is administered to a subject receiving an organ transplant to prevent occurrence of an autoimmune disease.

### Cryopreservation of Bone Marrow

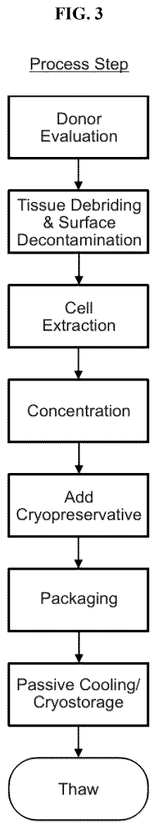

The present method provides a system for extracting and banking bone marrow for future clinical use according to the processing methods described above, as summarized in the flowchart of FIG. 3. This method can eliminate the failures of the current methods of matching bone marrow donors to groups that are tough to match, such as certain minorities. Once the bone marrow is cryopreserved and banked there is no uncertainty as to the source of the bone marrow, there is no wait for a future recipient and the bone marrow is available in large, repeatable volumes.

It is contemplated that each bone donor can yield three or more bags of bone marrow through the process described above, based on ten vertebrae and/or the ilium obtained from the donor. If at the end of the process for a given donor three bags of bone marrow are not obtained, the donor can be flagged as potentially not passing overall quality control. A predetermined volume of bone marrow in each bag is contemplated, such as 70 ml contained in 250 ml bags. This predetermined volume is used to calculate the volume of freeze media components necessary for efficient cryopreservation of the bone marrow pellet. The freeze media is a solution of a rinse media and a cryopreservation composition. The cryoprotectant composition can be a cell-permeable media, such as dimethyl sulfoxide (DMSO); 1, 2 propane diol; ethylene glycol; glycerol; foramamide; ethanediol or butane 2, 3 diol; and/or a non-permeable media, such as hydroxyethyl starch (HES), Dextran, sucrose, trehalose, lactose, raffinose, Ribotol, Mannitol or polyvinylpyrrolidone (PVP). HSA also provides cryoprotection through oncotic pressure, cell surface protein stabilization and reactive oxygen scavenging. In a preferred embodiment, the cryopreservation media is DMSO. The rinse media can be an electrolyte medium, such as PlasmaLyte, Isolyte, IMDM or other electrolyte solutions suitable for infusion. The freeze media can also include concentrations of oxyrase to reduce oxygen content to less than atmospheric, such as to less than 3% of atmospheric concentrations. The addition of oxyrase produces a hypobaric composition that can facilitate cryopreservation.

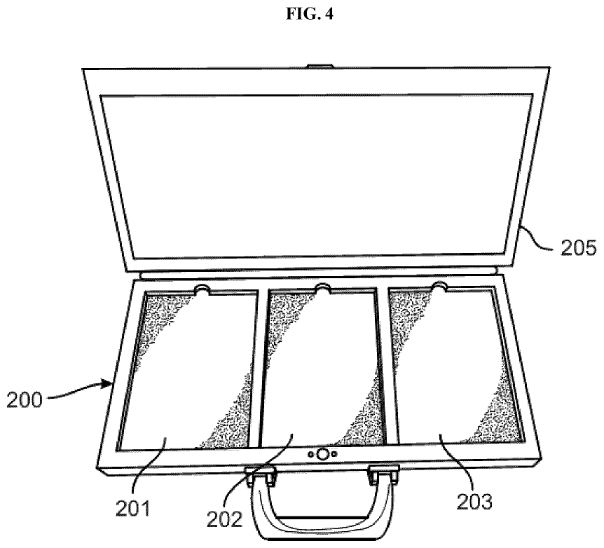

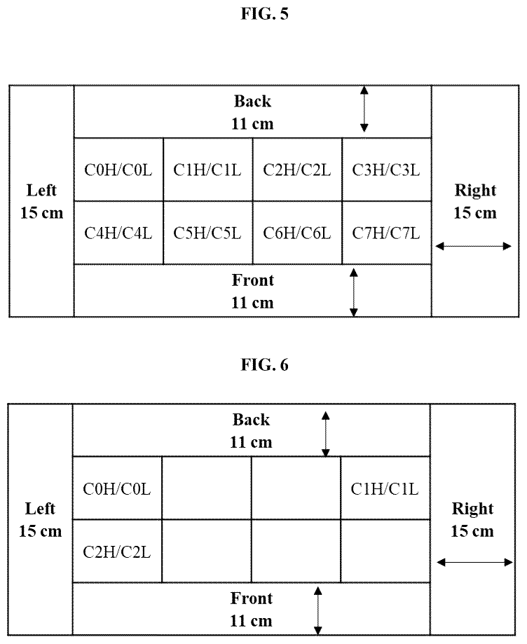

The freeze media is prepared by mixing the cryoprotectant and the rinse media according to the calculated total volume of freeze media needed for the volume of bone marrow collected. The bag containing the bone marrow is placed on a rocker for mixing and the freeze media is introduced into the bag by syringe. The freeze media is introduced at a particular rate over a predetermined time. In one embodiment, the freeze media is added at a rate of 10% of the media per minute, for a time of ten minutes. Once the media has been mixed with the concentrated bone marrow, a test sample is extracted by syringe. The remaining mixture of freeze media and bone marrow is injected in predetermined amounts into separate cryopreservation bags. In one embodiment, 70 ml of bone marrow mixture is introduced into each cryopreservation bag and air is drawn out with a syringe. At the end of the process, an 8 ml sample can be removed for sterility testing. Each cryopreservation bag is sealed to create four compartments, which are then separated for storage in cassettes to be stored in a cryo-freezer. In another embodiment, the separated compartments are stored in a passive cooling box, such as cooling box 200 shown in FIG. 4 or the cooling boxes described in U.S. Pat. No. 7,604,930, which is hereby incorporated by reference, in its entirety. A standard freezer box with or without a box rack may be used in these embodiments. In some embodiments, the cassettes are not stored in a passive cooling box. In some embodiments, the cassettes are arranged inside a cryo-freezer in a particular configuration to induce specific freezing rates. In some embodiments, the arrangement is the arrangement depicted in FIG. 5 or FIG. 6.

When the test samples from the particular bone marrow batch have been validated for cell count and sterility, the bags of cryopreserved bone marrow can be further cooled for long-term storage. In one embodiment, the bags are cooled at a controlled rate to prevent damage to the bone marrow and cells. An optimal cooling scheme to yield an optimal amount of viable bone marrow and cells comprises varying the cool rate at various stages of the cooling process. In some embodiments, the stages of the cooling process are referred to as “Supra-Freeze” (about 17° C. to the point of nucleation) and “Sub-Freeze” (from about −10° C. to −40° C.). Typically, nucleation occurs between about 7° C. and 15° C.

Aspects described in the present disclosure comprises a method for processing bone marrow or a derivative thereof (e.g. bone marrow derived cellular compositions), wherein the bone marrow or the derivative thereof is derived from a deceased donor, the method comprising: obtaining a bone or bone fragment from a deceased donor, optionally, processing the bone into bone fragments; extracting the bone marrow or the derivative thereof from the bone or bone fragment; and cryopreserving the bone marrow or the derivative thereof, wherein the cryopreserving comprises decreasing temperature of the bone marrow or the derivative thereof at a freeze rate of more than about −1° C./min in a static freezer. In some embodiments, the cryopreserving comprises cooling the bone marrow or the derivative thereof at a supra-freeze rate between about −2.5° C./min to about-5° C./min at least until at least one cell of the bone marrow or the derivative thereof is nucleated. In some embodiments, the cryopreserving comprises cooling the bone marrow or the derivative thereof at a supra-freeze rate between about −2.5° C./min to about −4° C./min at least until at least one cell of the bone marrow or the derivative thereof is nucleated. In some embodiments, the cryopreserving comprises cooling the bone marrow or the derivative thereof at a supra-freeze rate between about-2.5° C./min to about −3.5° C./min at least until at least one cell of the bone marrow or the derivative thereof is nucleated. In some embodiments, the cryopreserving comprises cooling the bone marrow or the derivative thereof at a sub-freeze rate between about −1° C./min to about −2° C./min. In some embodiments, the supra-freeze rate and the sub-freeze rate are maintained without the use of a passive cool box. In some embodiments, the cryopreserving comprises arranging one or more aliquots of the bone marrow or the derivative thereof inside the static freezer such that no aliquot contacts a wall of the static freezer. In some embodiments, the bone marrow or the derivative thereof comprises a population of CD34+ cells. In some embodiments, the population of CD34+ cells comprise at least 70% viable CD34+ cells after the bone marrow or the derivative thereof is thawed. In some embodiments, the population of CD34+ cells comprise at least 80% viable CD34+ cells after the bone marrow or the derivative thereof is thawed. In some embodiments, the static freezer is set at about −70° C. to −90° C. In some embodiments, the static freezer is set at −86° C. In some embodiments, the static freezer is set at −80° C.

In one specific embodiment, the bags are cooled at a rate of −1 to −40° C. per minute until the bags have reached a temperature suitable for plunging the bags into liquid nitrogen. A suitable temperature is in the range of −40 to −100° C. Once that temperature has been reached, the bags are cooled further at a more rapid rate to a temperature of below −130° C. for storage.

In some embodiments, the temperatures for freezing the bone marrow or bone marrow cells comprise the temperatures and freeze rates shown in Example 5. In some embodiments, the bone marrow or bone marrow cells can be cryopreserved at a supra-freeze rate or a supra-freeze range. In some embodiments, the bone marrow or bone marrow cells can be cryopreserved by freezing at both supra-freeze rate and sub-freeze rate. For example, the bone marrow or bone marrow cells can be cryopreserved by freezing at first with supra-freeze rate until a predetermined temperature is reached, which is then followed by switching freezing the bone marrow or bone marrow cells to a sub-freeze rate. In some embodiments, the nucleation temperature of the bone marrow or bone marrow cells can be reached during the supra-freeze. In some embodiments, the nucleation temperature of the bone marrow or bone marrow cells can be reached during the sub-freeze. In some embodiments, the nucleation temperature of the bone marrow or bone marrow cells can be reached during the switching between the supra-freeze and the sub-freeze.

In some instances, the bone marrow or bone marrow cells can be cryopreserved first with supra-freeze. For example, the bone marrow or bone marrow cells can be cryopreserved while the bone marrow or bone marrow cells are just processed and at room temperature. In some instances, the supra-freeze rate is generally higher (e.g. decreasing of the temperature at a faster rate) compared to the sub-freeze rate. In some embodiments, the supra-freeze rate is between about −6° C./min to about −0.5° C./min. In some embodiments, the supra-freeze rate is between about-0.5° C./min to about −1° C./min, about −0.5° C./min to about −1.5° C./min, about −0.5° C./min to about-2° C./min, about −0.5° C./min to about −2.5° C./min, about −0.5° C./min to about −3° C./min, about-0.5° C./min to about −3.5° C./min, about −0.5° C./min to about −4° C./min, about −0.5° C./min to about-4.5° C./min, about −0.5° C./min to about −5° C./min, about −0.5° C./min to about −5.5° C./min, about-0.5° C./min to about −6° C./min, about −1° C./min to about −1.5° C./min, about −1° C./min to about-2° C./min, about −1° C./min to about −2.5° C./min, about −1° C./min to about −3° C./min, about −1° C./min to about −3.5° C./min, about −1° C./min to about −4° C./min, about −1° C./min to about −4.5° C./min, about −1° C./min to about −5° C./min, about −1° C./min to about −5.5° C./min, about −1° C./min to about-6° C./min, about −1.5° C./min to about −2° C./min, about −1.5° C./min to about −2.5° C./min, about-1.5° C./min to about −3° C./min, about −1.5° C./min to about −3.5° C./min, about −1.5° C./min to about-4° C./min, about −1.5° C./min to about −4.5° C./min, about −1.5° C./min to about −5° C./min, about-1.5° C./min to about −5.5° C./min, about −1.5° C./min to about −6° C./min, about −2° C./min to about-2.5° C./min, about −2° C./min to about −3° C./min, about −2° C./min to about −3.5° C./min, about-2° C./min to about −4° C./min, about −2° C./min to about −4.5° C./min, about −2° C./min to about-5° C./min, about −2° C./min to about −5.5° C./min, about −2° C./min to about −6° C./min, about-2.5° C./min to about −3° C./min, about −2.5° C./min to about −3.5° C./min, about −2.5° C./min to about-4° C./min, about −2.5° C./min to about −4.5° C./min, about −2.5° C./min to about −5° C./min, about-2.5° C./min to about −5.5° C./min, about −2.5° C./min to about −6° C./min, about −3° C./min to about-3.5° C./min, about −3° C./min to about −4° C./min, about −3° C./min to about −4.5° C./min, about-3° C./min to about −5° C./min, about −3° C./min to about −5.5° C./min, about −3° C./min to about-6° C./min, about −3.5° C./min to about −4° C./min, about −3.5° C./min to about −4.5° C./min, about-3.5° C./min to about −5° C./min, about −3.5° C./min to about −5.5° C./min, about −3.5° C./min to about-6° C./min, about −4° C./min to about −4.5° C./min, about −4° C./min to about −5° C./min, about −4° C./min to about −5.5° C./min, about −4° C./min to about −6° C./min, about −4.5° C./min to about −5° C./min, about −4.5° C./min to about −5.5° C./min, about −4.5° C./min to about −6° C./min, about −5° C./min to about-5.5° C./min, about −5° C./min to about −6° C./min, or about −5.5° C./min to about −6° C./min. In some embodiments, the supra-freeze rate is between about −0.5° C./min, about −1° C./min, about-1.5° C./min, about −2° C./min, about −2.5° C./min, about −3° C./min, about −3.5° C./min, about −4° C./min, about −4.5° C./min, about −5° C./min, about −5.5° C./min, or about −6° C./min. In some embodiments, the supra-freeze rate is between at least about −0.5° C./min, about −1° C./min, about −1.5° C./min, about −2° C./min, about −2.5° C./min, about −3° C./min, about −3.5° C./min, about −4° C./min, about-4.5° C./min, about −5° C./min, or about −5.5° C./min. In some embodiments, the supra-freeze rate is between at most about −1° C./min, about −1.5° C./min, about −2° C./min, about −2.5° C./min, about-3° C./min, about −3.5° C./min, about −4° C./min, about −4.5° C./min, about −5° C./min, about −5.5° C./min, or about −6° C./min. In some embodiments, the supra-freeze rate was −3.2° C. In some embodiments, the supra-freeze rate is between about −2.54° C./min to −4.09° C./min.

In some embodiments, the bone marrow or bone marrow cells can be cryopreserved at a sub-freeze rate or a sub-freeze range. In some embodiments, the sub-freeze rate is between about −2.5° C./min to about −0.1° C./min. In some embodiments, the sub-freeze rate is between about-0.1° C./min to about −0.2° C./min, about −0.1° C./min to about −0.4° C./min, about −0.1° C./min to about −0.6° C./min, about −0.1° C./min to about −0.8° C./min, about −0.1° C./min to about −1° C./min, about-0.1° C./min to about −1.2° C./min, about −0.1° C./min to about −1.4° C./min, about −0.1° C./min to about −1.6° C./min, about −0.1° C./min to about −1.8° C./min, about −0.1° C./min to about −2° C./min, about-0.1° C./min to about −2.5° C./min, about −0.2° C./min to about −0.4° C./min, about −0.2° C./min to about −0.6° C./min, about −0.2° C./min to about −0.8° C./min, about −0.2° C./min to about −1° C./min, about-0.2° C./min to about −1.2° C./min, about −0.2° C./min to about −1.4° C./min, about −0.2° C./min to about −1.6° C./min, about −0.2° C./min to about −1.8° C./min, about −0.2° C./min to about −2° C./min, about-0.2° C./min to about −2.5° C./min, about −0.4° C./min to about −0.6° C./min, about −0.4° C./min to about −0.8° C./min, about −0.4° C./min to about −1° C./min, about −0.4° C./min to about −1.2° C./min, about-0.4° C./min to about −1.4° C./min, about −0.4° C./min to about −1.6° C./min, about −0.4° C./min to about −1.8° C./min, about −0.4° C./min to about −2° C./min, about −0.4° C./min to about −2.5° C./min, about-0.6° C./min to about −0.8° C./min, about −0.6° C./min to about −1° C./min, about −0.6° C./min to about-1.2° C./min, about −0.6° C./min to about −1.4° C./min, about −0.6° C./min to about −1.6° C./min, about-0.6° C./min to about −1.8° C./min, about −0.6° C./min to about −2° C./min, about −0.6° C./min to about-2.5° C./min, about −0.8° C./min to about −1° C./min, about −0.8° C./min to about −1.2° C./min, about-0.8° C./min to about −1.4° C./min, about −0.8° C./min to about −1.6° C./min, about −0.8° C./min to about −1.8° C./min, about −0.8° C./min to about −2° C./min, about −0.8° C./min to about −2.5° C./min, about-1° C./min to about −1.2° C./min, about −1° C./min to about −1.4° C./min, about −1° C./min to about-1.6° C./min, about −1° C./min to about −1.8° C./min, about −1° C./min to about −2° C./min, about-1° C./min to about −2.5° C./min, about −1.2° C./min to about −1.4° C./min, about −1.2° C./min to about-1.6° C./min, about −1.2° C./min to about −1.8° C./min, about −1.2° C./min to about −2° C./min, about-1.2° C./min to about −2.5° C./min, about −1.4° C./min to about −1.6° C./min, about −1.4° C./min to about −1.8° C./min, about −1.4° C./min to about −2° C./min, about −1.4° C./min to about −2.5° C./min, about-1.6° C./min to about −1.8° C./min, about −1.6° C./min to about −2° C./min, about −1.6° C./min to about-2.5° C./min, about −1.8° C./min to about −2° C./min, about −1.8° C./min to about −2.5° C./min, or about-2° C./min to about −2.5° C./min. In some embodiments, the sub-freeze rate is between about-0.1° C./min, about −0.2° C./min, about −0.4° C./min, about −0.6° C./min, about −0.8° C./min, about-1° C./min, about −1.2° C./min, about −1.4° C./min, about −1.6° C./min, about −1.8° C./min, about-2° C./min, or about −2.5° C./min. In some embodiments, the sub-freeze rate is between at least about −0.1° C./min, about −0.2° C./min, about −0.4° C./min, about −0.6° C./min, about −0.8° C./min, about-1° C./min, about −1.2° C./min, about −1.4° C./min, about −1.6° C./min, about −1.8° C./min, or about-2° C./min. In some embodiments, the sub-freeze rate is between at most about −0.2° C./min, about-0.4° C./min, about −0.6° C./min, about −0.8° C./min, about −1° C./min, about −1.2° C./min, about-1.4° C./min, about −1.6° C./min, about −1.8° C./min, about −2° C./min, or about −2.5° C./min. In some embodiments, the sub-freeze rate can be −1.36° C./min. In some embodiments, the sub-freeze rate comprises a range of −1.13° C./min to −1.62° C./min.

In some embodiments, the freeze rate for cryopreserving the bone marrow or the bone marrow cells described herein comprises determining the nucleation temperature. In some embodiments, the nucleation temperature is between about −24° C. to about −2° C. In some embodiments, the nucleation temperature is between about −2° C. to about −4° C., about −2° C. to about −6° C., about −2° C. to about −8° C., about −2° C. to about −10° C., about −2° C. to about −12° C., about −2° C. to about −14° C., about −2° C. to about −16° C., about −2° C. to about −18° C., about −2° C. to about −20° C., about −2° C. to about −22° C., about −2° C. to about −24° C., about −4° C. to about −6° C., about −4° C. to about −8° C., about −4° C. to about −10° C., about −4° C. to about −12° C., about −4° C. to about −14° C., about −4° C. to about −16° C., about −4° C. to about −18° C., about −4° C. to about −20° C., about −4° C. to about −22° C., about −4° C. to about −24° C., about −6° C. to about −8° C., about −6° C. to about −10° C., about −6° C. to about −12° C., about −6° C. to about −14° C., about −6° C. to about −16° C., about −6° C. to about −18° C., about −6° C. to about −20° C., about −6° C. to about −22° C., about −6° C. to about −24° C., about −8° C. to about −10° C., about −8° C. to about −12° C., about −8° C. to about −14° C., about −8° C. to about −16° C., about −8° C. to about −18° C., about −8° C. to about −20° C., about −8° C. to about −22° C., about −8° C. to about −24° C., about −10° C. to about −12° C., about −10° C. to about −14° C., about −10° C. to about −16° C., about −10° C. to about −18° C., about −10° C. to about −20° C., about −10° C. to about-22° C., about −10° C. to about −24° C., about −12° C. to about −14° C., about −12° C. to about −16° C., about −12° C. to about −18° C., about −12° C. to about −20° C., about −12° C. to about −22° C., about −12° C. to about −24° C., about −14° C. to about −16° C., about −14° C. to about −18° C., about −14° C. to about −20° C., about −14° C. to about −22° C., about −14° C. to about −24° C., about −16° C. to about −18° C., about −16° C. to about −20° C., about −16° C. to about −22° C., about −16° C. to about −24° C., about −18° C. to about-20° C., about −18° C. to about −22° C., about −18° C. to about −24° C., about −20° C. to about −22° C., about −20° C. to about −24° C., or about −22° C. to about −24° C. In some embodiments, the nucleation temperature is between about −2° C., about −4° C., about −6° C., about −8° C., about −10° C., about −12° C., about −14° C., about −16° C., about −18° C., about −20° C., about −22° C., or about −24° C. In some embodiments, the nucleation temperature is between at least about −2° C., about −4° C., about −6° C., about −8° C., about −10° C., about −12° C., about −14° C., about −16° C., about −18° C., about −20° C., or about −22° C. In some embodiments, the nucleation temperature is between at most about −4° C., about −6° C., about −8° C., about −10° C., about −12° C., about −14° C., about −16° C., about −18° C., about −20° C., about −22° C., or about −24° C. In some embodiments, the nucleation temperature can be about −12.31° C./min. In some embodiments, the nucleation temperature can comprise a range of between about −7.24° C. to −17.52° C.

In some embodiments, the bone marrow or bone marrow cells to be cryopreserved can be placed in a container or bag such as a cryobag. In some cases, the cryobag can be subsequently placed into a cooling box for freezing. Alternative, the cryobag is not placed in a cooling box. In some cases, the cryobag can be placed in a cassette and the subsequently placed in a freezing environment (e.g. placed in a freezer). In some cases, the cryobag can be placed in a freezing environment of liquid nitrogen or vapor stemmed from liquid nitrogen. In some cases, the cryobag can be placed in different compartments or different levels of shelfs in the freezer or the in the liquid nitrogen or liquid nitrogen vapor. In some embodiments, the cryobag containing the bone marrow or bone marrow cells can be placed in a position as depicted in FIG. 5 or FIG. 6.

A cryopreservation bag is placed within a corresponding compartment 201-203 of the cooling box 200 and the overlapping cover 205 is closed over the compartments to provide a sealed environment for cryo-preservation of the contents of the bags. The cooling box is placed within a cryo freezer such that the cooling box produces a cooling rate of −0.5 to −2° C./min, and typically of −1° C./min, with nucleation temperatures above −20° C. The freezing process continues at the prescribed rate until the temperature of the bone marrow reaches a suitable temperature. The suitable temperature for storage of the bags is a temperature <−80° C. or <−150° C.

In another embodiment, the bags are cooled in a static chamber temperature as opposed to the controlled rate cryopreservation described above. In the passive cooling approach, the cooling box is placed in a −86° C. freezer until the bags reach a stable temperature. In some cases, the freezer can be set at a range of temperature from about −100° C. to about −60° C. In some cases, the freezer can be set at a range of temperature from about −60° C. to about −65° C., about −60° C. to about −70° C., about −60° C. to about −75° C., about −60° C. to about −80° C., about −60° C. to about-82° C., about −60° C. to about −84° C., about −60° C. to about −86° C., about −60° C. to about −88° C., about −60° C. to about −90° C., about −60° C. to about −95° C., about −60° C. to about −100° C., about −65° C. to about −70° C., about −65° C. to about −75° C., about −65° C. to about −80° C., about −65° C. to about-82° C., about −65° C. to about −84° C., about −65° C. to about −86° C., about −65° C. to about −88° C., about −65° C. to about −90° C., about −65° C. to about −95° C., about −65° C. to about −100° C., about −70° C. to about −75° C., about −70° C. to about −80° C., about −70° C. to about −82° C., about −70° C. to about-84° C., about −70° C. to about −86° C., about −70° C. to about −88° C., about −70° C. to about −90° C., about −70° C. to about −95° C., about −70° C. to about −100° C., about −75° C. to about −80° C., about −75° C. to about −82° C., about −75° C. to about −84° C., about −75° C. to about −86° C., about −75° C. to about-88° C., about −75° C. to about −90° C., about −75° C. to about −95° C., about −75° C. to about −100° C., about −80° C. to about −82° C., about −80° C. to about −84° C., about −80° C. to about −86° C., about −80° C. to about −88° C., about −80° C. to about −90° C., about −80° C. to about −95° C., about −80° C. to about-100° C., about −82° C. to about −84° C., about −82° C. to about −86° C., about −82° C. to about −88° C., about −82° C. to about −90° C., about −82° C. to about −95° C., about −82° C. to about −100° C., about-84° C. to about −86° C., about −84° C. to about −88° C., about −84° C. to about −90° C., about −84° C. to about −95° C., about −84° C. to about −100° C., about −86° C. to about −88° C., about −86° C. to about-90° C., about −86° C. to about −95° C., about −86° C. to about −100° C., about −88° C. to about −90° C., about −88° C. to about −95° C., about −88° C. to about −100° C., about −90° C. to about −95° C., about-90° C. to about −100° C., or about −95° C. to about −100° C. In some cases, the freezer can be set at a range of temperature from about −60° C., about −65° C., about −70° C., about −75° C., about −80° C., about −82° C., about −84° C., about −86° C., about −88° C., about −90° C., about −95° C., or about −100° C. In some cases, the freezer can be set at a range of temperature from at least about −60° C., about-65° C., about −70° C., about −75° C., about −80° C., about −82° C., about −84° C., about −86° C., about-88° C., about −90° C., or about −95° C. In some cases, the freezer can be set at a range of temperature from at most about −65° C., about −70° C., about −75° C., about −80° C., about −82° C., about −84° C., about −86° C., about −88° C., about −90° C., about −95° C., or about −100° C.

It is contemplated that the cryopreservation storage can be in many forms. For instance, the cryopreserved bone marrow can be contained in bags of 1 ml to 5 ml volume or vials of 0.1 to 15 ml volumes. In a preferred embodiment, the bags with 70 ml bone marrow are stored in a cooling box within a cryogenic freezer.

### Cryobanking

The cryopreserved bone marrow is cryobanked for later thawing and extraction of desired cells. The thawed bone marrow can be provided for a wide range of treatments including treatment for leukemias, brain tumors, breast cancer, Hodgkin's disease, multiple myeloma, neuroblastoma, non-Hodgkin's lymphoma, blood cancers, ovarian cancer, sarcoma, testicular cancer, other solid organ cancer, rheumatoid arthritis, multiple sclerosis, diabetes mellitus, cystic fibrosus, Alzheimer's disease, genetic immunodeficiencies, metabolic disorders, marrow failure syndromes, and HIV. Bone marrow can also be used for induction of immunotolerance to reduce the potential rejection of an implant obtained from an organ donor. Bone marrow treatments can also be indicated for casualties caused by radiation and certain biological weapons.

Another aspect of the present disclosure comprises a method for processing a biological sample comprising cells or a derivative thereof, the method comprising: generating a first volume of the biological sample comprising cells or a derivative thereof, wherein the first volume comprises a first concentration of cells or a derivative thereof; generating a second volume of the biological sample comprising cells or a derivative thereof, wherein the second volume is less than the first volume and comprises a second concentration of the cells wherein the second concentration of the cells is no more than 30% different than the first concentration of the cells; and cooling the first volume at a first cooling rate and cooling the second volume at a second cooling rate, wherein the first cooling rate is faster than the second cooling rate; wherein a post-thaw cell proliferation rate of the cells in the first volume is no more than 30% different than a post-thaw proliferation rate of the cells in the second volume. In some embodiments, the first volume is contained in a first container, wherein the second volume is contained in a second container, and wherein the first container and the second container are exposed to a common temperature.

In some embodiments, the second volume is less than about 0.5% of the first volume to about 50% of the first volume. In some embodiments, the second volume is less than about 50% of the first volume to about 40% of the first volume, about 50% of the first volume to about 30% of the first volume, about 50% of the first volume to about 20% of the first volume, about 50% of the first volume to about 10% of the first volume, about 50% of the first volume to about 5% of the first volume, about 50% of the first volume to about 1% of the first volume, about 50% of the first volume to about 0.5% of the first volume, about 40% of the first volume to about 30% of the first volume, about 40% of the first volume to about 20% of the first volume, about 40% of the first volume to about 10% of the first volume, about 40% of the first volume to about 5% of the first volume, about 40% of the first volume to about 1% of the first volume, about 40% of the first volume to about 0.5% of the first volume, about 30% of the first volume to about 20% of the first volume, about 30% of the first volume to about 10% of the first volume, about 30% of the first volume to about 5% of the first volume, about 30% of the first volume to about 1% of the first volume, about 30% of the first volume to about 0.5% of the first volume, about 20% of the first volume to about 10% of the first volume, about 20% of the first volume to about 5% of the first volume, about 20% of the first volume to about 1% of the first volume, about 20% of the first volume to about 0.5% of the first volume, about 10% of the first volume to about 5% of the first volume, about 10% of the first volume to about 1% of the first volume, about 10% of the first volume to about 0.5% of the first volume, about 5% of the first volume to about 1% of the first volume, about 5% of the first volume to about 0.5% of the first volume, or about 1% of the first volume to about 0.5% of the first volume. In some embodiments, the second volume is less than about 50% of the first volume, about 40% of the first volume, about 30% of the first volume, about 20% of the first volume, about 10% of the first volume, about 5% of the first volume, about 1% of the first volume, or about 0.5% of the first volume. In some embodiments, the second volume is less than at least about 50% of the first volume, about 40% of the first volume, about 30% of the first volume, about 20% of the first volume, about 10% of the first volume, about 5% of the first volume, or about 1% of the first volume. In some embodiments, the second volume is less than at most about 40% of the first volume, about 30% of the first volume, about 20% of the first volume, about 10% of the first volume, about 5% of the first volume, about 1% of the first volume, or about 0.5% of the first volume. In some embodiments, the second volume is less than 50% of the first volume. In some embodiments, the second volume is less than 40% of the first volume. In some embodiments, the second volume is less than 37.5% of the first volume. In some embodiments, the second volume is less than 35% of the first volume. In some embodiments, the second volume is less than 30% of the first volume. In some embodiments, the second volume is less than 20% of the first volume. In some embodiments, the second volume is less than 15% of the first volume. In some embodiments, the second volume is less than 10% of the first volume. In some embodiments, the second volume is less than 5% of the first volume. In some embodiments, the second volume is less than 1% of the first volume.

In some embodiments, a post-thaw viability rate of the cells in the first volume is no more than about 0.5% different than a post-thaw viability rate of the cells in the second volume to about 30% different than a post-thaw viability rate of the cells in the second volume. In some embodiments, a post-thaw viability rate of the cells in the first volume is no more than about 30% different than a post-thaw viability rate of the cells in the second volume to about 25% different than a post-thaw viability rate of the cells in the second volume, about 30% different than a post-thaw viability rate of the cells in the second volume to about 20% different than a post-thaw viability rate of the cells in the second volume, about 30% different than a post-thaw viability rate of the cells in the second volume to about 15% different than a post-thaw viability rate of the cells in the second volume, about 30% different than a post-thaw viability rate of the cells in the second volume to about 10% different than a post-thaw viability rate of the cells in the second volume, about 30% different than a post-thaw viability rate of the cells in the second volume to about 5% different than a post-thaw viability rate of the cells in the second volume, about 30% different than a post-thaw viability rate of the cells in the second volume to about 1% different than a post-thaw viability rate of the cells in the second volume, about 30% different than a post-thaw viability rate of the cells in the second volume to about 0.5% different than a post-thaw viability rate of the cells in the second volume, about 25% different than a post-thaw viability rate of the cells in the second volume to about 20% different than a post-thaw viability rate of the cells in the second volume, about 25% different than a post-thaw viability rate of the cells in the second volume to about 15% different than a post-thaw viability rate of the cells in the second volume, about 25% different than a post-thaw viability rate of the cells in the second volume to about 10% different than a post-thaw viability rate of the cells in the second volume, about 25% different than a post-thaw viability rate of the cells in the second volume to about 5% different than a post-thaw viability rate of the cells in the second volume, about 25% different than a post-thaw viability rate of the cells in the second volume to about 1% different than a post-thaw viability rate of the cells in the second volume, about 25% different than a post-thaw viability rate of the cells in the second volume to about 0.5% different than a post-thaw viability rate of the cells in the second volume, about 20% different than a post-thaw viability rate of the cells in the second volume to about 15% different than a post-thaw viability rate of the cells in the second volume, about 20% different than a post-thaw viability rate of the cells in the second volume to about 10% different than a post-thaw viability rate of the cells in the second volume, about 20% different than a post-thaw viability rate of the cells in the second volume to about 5% different than a post-thaw viability rate of the cells in the second volume, about 20% different than a post-thaw viability rate of the cells in the second volume to about 1% different than a post-thaw viability rate of the cells in the second volume, about 20% different than a post-thaw viability rate of the cells in the second volume to about 0.5% different than a post-thaw viability rate of the cells in the second volume, about 15% different than a post-thaw viability rate of the cells in the second volume to about 10% different than a post-thaw viability rate of the cells in the second volume, about 15% different than a post-thaw viability rate of the cells in the second volume to about 5% different than a post-thaw viability rate of the cells in the second volume, about 15% different than a post-thaw viability rate of the cells in the second volume to about 1% different than a post-thaw viability rate of the cells in the second volume, about 15% different than a post-thaw viability rate of the cells in the second volume to about 0.5% different than a post-thaw viability rate of the cells in the second volume, about 10% different than a post-thaw viability rate of the cells in the second volume to about 5% different than a post-thaw viability rate of the cells in the second volume, about 10% different than a post-thaw viability rate of the cells in the second volume to about 1% different than a post-thaw viability rate of the cells in the second volume, about 10% different than a post-thaw viability rate of the cells in the second volume to about 0.5% different than a post-thaw viability rate of the cells in the second volume, about 5% different than a post-thaw viability rate of the cells in the second volume to about 1% different than a post-thaw viability rate of the cells in the second volume, about 5% different than a post-thaw viability rate of the cells in the second volume to about 0.5% different than a post-thaw viability rate of the cells in the second volume, or about 1% different than a post-thaw viability rate of the cells in the second volume to about 0.5% different than a post-thaw viability rate of the cells in the second volume. In some embodiments, a post-thaw viability rate of the cells in the first volume is no more than about 30% different than a post-thaw viability rate of the cells in the second volume, about 25% different than a post-thaw viability rate of the cells in the second volume, about 20% different than a post-thaw viability rate of the cells in the second volume, about 15% different than a post-thaw viability rate of the cells in the second volume, about 10% different than a post-thaw viability rate of the cells in the second volume, about 5% different than a post-thaw viability rate of the cells in the second volume, about 1% different than a post-thaw viability rate of the cells in the second volume, or about 0.5% different than a post-thaw viability rate of the cells in the second volume. In some embodiments, a post-thaw viability rate of the cells in the first volume is no more than at least about 30% different than a post-thaw viability rate of the cells in the second volume, about 25% different than a post-thaw viability rate of the cells in the second volume, about 20% different than a post-thaw viability rate of the cells in the second volume, about 15% different than a post-thaw viability rate of the cells in the second volume, about 10% different than a post-thaw viability rate of the cells in the second volume, about 5% different than a post-thaw viability rate of the cells in the second volume, or about 1% different than a post-thaw viability rate of the cells in the second volume. In some embodiments, a post-thaw viability rate of the cells in the first volume is no more than at most about 25% different than a post-thaw viability rate of the cells in the second volume, about 20% different than a post-thaw viability rate of the cells in the second volume, about 15% different than a post-thaw viability rate of the cells in the second volume, about 10% different than a post-thaw viability rate of the cells in the second volume, about 5% different than a post-thaw viability rate of the cells in the second volume, about 1% different than a post-thaw viability rate of the cells in the second volume, or about 0.5% different than a post-thaw viability rate of the cells in the second volume. In some embodiments, a post-thaw viability rate of the cells in the first volume is no more than 30% different than a post-thaw viability rate of the cells in the second volume. In some embodiments, a post-thaw viability rate of the cells in the first volume is no more than 25% different than a post-thaw viability rate of the cells in the second volume. In some embodiments, a post-thaw viability rate of the cells in the first volume is no more than 20% different than a post-thaw viability rate of the cells in the second volume. In some embodiments, a post-thaw viability rate of the cells in the first volume is no more than 15% different than a post-thaw viability rate of the cells in the second volume. In some embodiments, a post-thaw viability rate of the cells in the first volume is no more than 13.6% different than a post-thaw viability rate of the cells in the second volume. In some embodiments, a post-thaw viability rate of the cells in the first volume is no more than 10% different than a post-thaw viability rate of the cells in the second volume. In some embodiments, a post-thaw viability rate of the cells in the first volume is no more than 5% different than a post-thaw viability rate of the cells in the second volume. In some embodiments, a post-thaw cell proliferation rate of the cells in the first volume is no more than 25% different than a post-thaw proliferation rate of the cells in the second volume. In some embodiments, a post-thaw cell proliferation rate of the cells in the first volume is no more than 20% different than a post-thaw proliferation rate of the cells in the second volume. In some embodiments, a post-thaw cell proliferation rate of the cells in the first volume is no more than 15% different than a post-thaw proliferation rate of the cells in the second volume. In some embodiments, a post-thaw cell proliferation rate of the cells in the first volume is no more than 13.6% different than a post-thaw proliferation rate of the cells in the second volume. In some embodiments, a post-thaw cell proliferation rate of the cells in the first volume is no more than 10% different than a post-thaw proliferation rate of the cells in the second volume. In some embodiments, a post-thaw cell proliferation rate of the cells in the first volume is no more than 5% different than a post-thaw proliferation rate of the cells in the second volume. In some embodiments, the post-thaw viability rate of the cells is at least 50%.

In some embodiments, the post-thaw proliferation rate of the cells is at least 1 CFU-GM/10⁵ cells. In some embodiments, the post-thaw proliferation rate of the cells is at least about 1 CFU-GM/10⁵ cells to about 200 CFU-GM/10⁵ cells. In some embodiments, the post-thaw proliferation rate of the cells is at least about 1 CFU-GM/10⁵ cells to about 10 CFU-GM/10⁵ cells, about 1 CFU-GM/10⁵ cells to about 20 CFU-GM/10⁵ cells, about 1 CFU-GM/10⁵ cells to about 30 CFU-GM/10⁵ cells, about 1 CFU-GM/10⁵ cells to about 40 CFU-GM/10⁵ cells, about 1 CFU-GM/10⁵ cells to about 50 CFU-GM/10⁵ cells, about 1 CFU-GM/10⁵ cells to about 60 CFU-GM/10⁵ cells, about 1 CFU-GM/10⁵ cells to about 70 CFU-GM/10⁵ cells, about 1 CFU-GM/10⁵ cells to about 80 CFU-GM/10⁵ cells, about 1 CFU-GM/10⁵ cells to about 90 CFU-GM/10⁵ cells, about 1 CFU-GM/10⁵ cells to about 100 CFU-GM/10⁵ cells, about 1 CFU-GM/10⁵ cells to about 200 CFU-GM/10⁵ cells, about 10 CFU-GM/10⁵ cells to about 20 CFU-GM/10⁵ cells, about 10 CFU-GM/10⁵ cells to about 30 CFU-GM/10⁵ cells, about 10 CFU-GM/10⁵ cells to about 40 CFU-GM/10⁵ cells, about 10 CFU-GM/10⁵ cells to about 50 CFU-GM/10⁵ cells, about 10 CFU-GM/10⁵ cells to about 60 CFU-GM/10⁵ cells, about 10 CFU-GM/10⁵ cells to about 70 CFU-GM/10⁵ cells, about 10 CFU-GM/10⁵ cells to about 80 CFU-GM/10⁵ cells, about 10 CFU-GM/10⁵ cells to about 90 CFU-GM/10⁵ cells, about 10 CFU-GM/10⁵ cells to about 100 CFU-GM/10⁵ cells, about 10 CFU-GM/10⁵ cells to about 200 CFU-GM/10⁵ cells, about 20 CFU-GM/10⁵ cells to about 30 CFU-GM/10⁵ cells, about 20 CFU-GM/10⁵ cells to about 40 CFU-GM/10⁵ cells, about 20 CFU-GM/10⁵ cells to about 50 CFU-GM/10⁵ cells, about 20 CFU-GM/10⁵ cells to about 60 CFU-GM/10⁵ cells, about 20 CFU-GM/10⁵ cells to about 70 CFU-GM/10⁵ cells, about 20 CFU-GM/10⁵ cells to about 80 CFU-GM/10⁵ cells, about 20 CFU-GM/10⁵ cells to about 90 CFU-GM/10⁵ cells, about 20 CFU-GM/10⁵ cells to about 100 CFU-GM/10⁵ cells, about 20 CFU-GM/10⁵ cells to about 200 CFU-GM/10⁵ cells, about 30 CFU-GM/10⁵ cells to about 40 CFU-GM/10⁵ cells, about 30 CFU-GM/10⁵ cells to about 50 CFU-GM/10⁵ cells, about 30 CFU-GM/10⁵ cells to about 60 CFU-GM/10⁵ cells, about 30 CFU-GM/10⁵ cells to about 70 CFU-GM/10⁵ cells, about 30 CFU-GM/10⁵ cells to about 80 CFU-GM/10⁵ cells, about 30 CFU-GM/10⁵ cells to about 90 CFU-GM/10⁵ cells, about 30 CFU-GM/10⁵ cells to about 100 CFU-GM/10⁵ cells, about 30 CFU-GM/10⁵ cells to about 200 CFU-GM/10⁵ cells, about 40 CFU-GM/10⁵ cells to about 50 CFU-GM/10⁵ cells, about 40 CFU-GM/10⁵ cells to about 60 CFU-GM/10⁵ cells, about 40 CFU-GM/10⁵ cells to about 70 CFU-GM/10⁵ cells, about 40 CFU-GM/10⁵ cells to about 80 CFU-GM/10⁵ cells, about 40 CFU-GM/10⁵ cells to about 90 CFU-GM/10⁵ cells, about 40 CFU-GM/10⁵ cells to about 100 CFU-GM/10⁵ cells, about 40 CFU-GM/10⁵ cells to about 200 CFU-GM/10⁵ cells, about 50 CFU-GM/10⁵ cells to about 60 CFU-GM/10⁵ cells, about 50 CFU-GM/10⁵ cells to about 70 CFU-GM/10⁵ cells, about 50 CFU-GM/10⁵ cells to about 80 CFU-GM/10⁵ cells, about 50 CFU-GM/10⁵ cells to about 90 CFU-GM/10⁵ cells, about 50 CFU-GM/10⁵ cells to about 100 CFU-GM/10⁵ cells, about 50 CFU-GM/10⁵ cells to about 200 CFU-GM/10⁵ cells, about 60 CFU-GM/10⁵ cells to about 70 CFU-GM/10⁵ cells, about 60 CFU-GM/10⁵ cells to about 80 CFU-GM/10⁵ cells, about 60 CFU-GM/10⁵ cells to about 90 CFU-GM/10⁵ cells, about 60 CFU-GM/10⁵ cells to about 100 CFU-GM/10⁵ cells, about 60 CFU-GM/10⁵ cells to about 200 CFU-GM/10⁵ cells, about 70 CFU-GM/10⁵ cells to about 80 CFU-GM/10⁵ cells, about 70 CFU-GM/10⁵ cells to about 90 CFU-GM/10⁵ cells, about 70 CFU-GM/10⁵ cells to about 100 CFU-GM/10⁵ cells, about 70 CFU-GM/10⁵ cells to about 200 CFU-GM/10⁵ cells, about 80 CFU-GM/10⁵ cells to about 90 CFU-GM/10⁵ cells, about 80 CFU-GM/10⁵ cells to about 100 CFU-GM/10⁵ cells, about 80 CFU-GM/10⁵ cells to about 200 CFU-GM/10⁵ cells, about 90 CFU-GM/10⁵ cells to about 100 CFU-GM/10⁵ cells, about 90 CFU-GM/10⁵ cells to about 200 CFU-GM/10⁵ cells, or about 100 CFU-GM/10⁵ cells to about 200 CFU-GM/10⁵ cells. In some embodiments, the post-thaw proliferation rate of the cells is at least about 1 CFU-GM/10⁵ cells, about 10 CFU-GM/10⁵ cells, about 20 CFU-GM/10⁵ cells, about 30 CFU-GM/10⁵ cells, about 40 CFU-GM/10⁵ cells, about 50 CFU-GM/10⁵ cells, about 60 CFU-GM/10⁵ cells, about 70 CFU-GM/10⁵ cells, about 80 CFU-GM/10⁵ cells, about 90 CFU-GM/10⁵ cells, about 100 CFU-GM/10⁵ cells, or about 200 CFU-GM/10⁵ cells. In some embodiments, the post-thaw proliferation rate of the cells is at least at least about 1 CFU-GM/10⁵ cells, about 10 CFU-GM/10⁵ cells, about 20 CFU-GM/10⁵ cells, about 30 CFU-GM/10⁵ cells, about 40 CFU-GM/10⁵ cells, about 50 CFU-GM/10⁵ cells, about 60 CFU-GM/10⁵ cells, about 70 CFU-GM/10⁵ cells, about 80 CFU-GM/10⁵ cells, about 90 CFU-GM/10⁵ cells, or about 100 CFU-GM/10⁵ cells. In some embodiments, the post-thaw proliferation rate of the cells is at least at most about 10 CFU-GM/10⁵ cells, about 20 CFU-GM/10⁵ cells, about 30 CFU-GM/10⁵ cells, about 40 CFU-GM/10⁵ cells, about 50 CFU-GM/10⁵ cells, about 60 CFU-GM/10⁵ cells, about 70 CFU-GM/10⁵ cells, about 80 CFU-GM/10⁵ cells, about 90 CFU-GM/10⁵ cells, about 100 CFU-GM/10⁵ cells, or about 200 CFU-GM/10⁵ cells.

In some embodiments, the first cooling rate and the second cooling rate comprise a supra-freeze rate between about 0° C./min to about −5° C./min at least until ice has nucleated in a freezing medium. In some instances, the biological sample or derivative thereof can be cryopreserved first with supra-freeze. For example, the biological sample or derivative thereof can be cryopreserved while the biological sample or derivative thereof are just processed and at room temperature. In some instances, the supra-freeze rate is generally higher (e.g. decreasing of the temperature at a faster rate) compared to the sub-freeze rate. In some embodiments, the supra-freeze rate is between about −6° C./min to about −0.5° C./min. In some embodiments, the supra-freeze rate is between about −0.5° C./min to about −1° C./min, about −0.5° C./min to about −1.5° C./min, about −0.5° C./min to about −2° C./min, about −0.5° C./min to about −2.5° C./min, about −0.5° C./min to about-3° C./min, about −0.5° C./min to about −3.5° C./min, about −0.5° C./min to about −4° C./min, about-0.5° C./min to about −4.5° C./min, about −0.5° C./min to about −5° C./min, about −0.5° C./min to about-5.5° C./min, about −0.5° C./min to about −6° C./min, about −1° C./min to about −1.5° C./min, about-1° C./min to about −2° C./min, about −1° C./min to about −2.5° C./min, about −1° C./min to about-3° C./min, about −1° C./min to about −3.5° C./min, about −1° C./min to about −4° C./min, about −1° C./min to about −4.5° C./min, about −1° C./min to about −5° C./min, about −1° C./min to about −5.5° C./min, about −1° C./min to about −6° C./min, about −1.5° C./min to about −2° C./min, about −1.5° C./min to about-2.5° C./min, about −1.5° C./min to about −3° C./min, about −1.5° C./min to about −3.5° C./min, about-1.5° C./min to about −4° C./min, about −1.5° C./min to about −4.5° C./min, about −1.5° C./min to about-5° C./min, about −1.5° C./min to about −5.5° C./min, about −1.5° C./min to about −6° C./min, about-2° C./min to about −2.5° C./min, about −2° C./min to about −3° C./min, about −2° C./min to about-3.5° C./min, about −2° C./min to about −4° C./min, about −2° C./min to about −4.5° C./min, about-2° C./min to about −5° C./min, about −2° C./min to about −5.5° C./min, about −2° C./min to about-6° C./min, about −2.5° C./min to about −3° C./min, about −2.5° C./min to about −3.5° C./min, about-2.5° C./min to about −4° C./min, about −2.5° C./min to about −4.5° C./min, about −2.5° C./min to about-5° C./min, about −2.5° C./min to about −5.5° C./min, about −2.5° C./min to about −6° C./min, about-3° C./min to about −3.5° C./min, about −3° C./min to about −4° C./min, about −3° C./min to about-4.5° C./min, about −3° C./min to about −5° C./min, about −3° C./min to about −5.5° C./min, about-3° C./min to about −6° C./min, about −3.5° C./min to about −4° C./min, about −3.5° C./min to about-4.5° C./min, about −3.5° C./min to about −5° C./min, about −3.5° C./min to about −5.5° C./min, about-3.5° C./min to about −6° C./min, about −4° C./min to about −4.5° C./min, about −4° C./min to about-5° C./min, about −4° C./min to about −5.5° C./min, about −4° C./min to about −6° C./min, about-4.5° C./min to about −5° C./min, about −4.5° C./min to about −5.5° C./min, about −4.5° C./min to about-6° C./min, about −5° C./min to about −5.5° C./min, about −5° C./min to about −6° C./min, or about-5.5° C./min to about −6° C./min. In some embodiments, the supra-freeze rate is between about-0.5° C./min, about −1° C./min, about −1.5° C./min, about −2° C./min, about −2.5° C./min, about −3° C./min, about −3.5° C./min, about −4° C./min, about −4.5° C./min, about −5° C./min, about −5.5° C./min, or about −6° C./min. In some embodiments, the supra-freeze rate is between at least about −0.5° C./min, about −1° C./min, about −1.5° C./min, about −2° C./min, about −2.5° C./min, about −3° C./min, about-3.5° C./min, about −4° C./min, about −4.5° C./min, about −5° C./min, or about −5.5° C./min. In some embodiments, the supra-freeze rate is between at most about −1° C./min, about −1.5° C./min, about-2° C./min, about −2.5° C./min, about −3° C./min, about −3.5° C./min, about −4° C./min, about −4.5° C./min, about −5° C./min, about −5.5° C./min, or about −6° C./min. In some embodiments, the supra-freeze rate was −3.2° C. In some embodiments, the supra-freeze rate is between about −2.54° C./min to −4.09° C./min. In some embodiments, the first cooling rate and the second cooling rate comprise a supra-freeze rate between about −2.5° C./min to about −4° C./min at least until ice has nucleated in a freezing medium. In some embodiments, the first cooling rate and the second cooling rate comprise a supra-freeze rate between about −2.5° C./min to about −3.5° C./min at least until ice has nucleated in a freezing medium.

In some embodiments, the first cooling rate and the second cooling rate comprise a sub-freeze rate between about −1° C./min to about −2° C./min. In some embodiments, the sub-freeze rate is between about −2.5° C./min to about −0.1° C./min. In some embodiments, the sub-freeze rate is between about −0.1° C./min to about −0.2° C./min, about −0.1° C./min to about −0.4° C./min, about-0.1° C./min to about −0.6° C./min, about −0.1° C./min to about −0.8° C./min, about −0.1° C./min to about-1° C./min, about −0.1° C./min to about −1.2° C./min, about −0.1° C./min to about −1.4° C./min, about-0.1° C./min to about −1.6° C./min, about −0.1° C./min to about −1.8° C./min, about −0.1° C./min to about-2° C./min, about −0.1° C./min to about −2.5° C./min, about −0.2° C./min to about −0.4° C./min, about-0.2° C./min to about −0.6° C./min, about −0.2° C./min to about −0.8° C./min, about −0.2° C./min to about-1° C./min, about −0.2° C./min to about −1.2° C./min, about −0.2° C./min to about −1.4° C./min, about-0.2° C./min to about −1.6° C./min, about −0.2° C./min to about −1.8° C./min, about −0.2° C./min to about-2° C./min, about −0.2° C./min to about −2.5° C./min, about −0.4° C./min to about −0.6° C./min, about-0.4° C./min to about −0.8° C./min, about −0.4° C./min to about −1° C./min, about −0.4° C./min to about-1.2° C./min, about −0.4° C./min to about −1.4° C./min, about −0.4° C./min to about −1.6° C./min, about-0.4° C./min to about −1.8° C./min, about −0.4° C./min to about −2° C./min, about −0.4° C./min to about-2.5° C./min, about −0.6° C./min to about −0.8° C./min, about −0.6° C./min to about −1° C./min, about-0.6° C./min to about −1.2° C./min, about −0.6° C./min to about −1.4° C./min, about −0.6° C./min to about-1.6° C./min, about −0.6° C./min to about −1.8° C./min, about −0.6° C./min to about −2° C./min, about-0.6° C./min to about −2.5° C./min, about −0.8° C./min to about −1° C./min, about −0.8° C./min to about-1.2° C./min, about −0.8° C./min to about −1.4° C./min, about −0.8° C./min to about −1.6° C./min, about-0.8° C./min to about −1.8° C./min, about −0.8° C./min to about −2° C./min, about −0.8° C./min to about-2.5° C./min, about −1° C./min to about −1.2° C./min, about −1° C./min to about −1.4° C./min, about-1° C./min to about −1.6° C./min, about −1° C./min to about −1.8° C./min, about −1° C./min to about-2° C./min, about −1° C./min to about −2.5° C./min, about −1.2° C./min to about −1.4° C./min, about-1.2° C./min to about −1.6° C./min, about −1.2° C./min to about −1.8° C./min, about −1.2° C./min to about-2° C./min, about −1.2° C./min to about −2.5° C./min, about −1.4° C./min to about −1.6° C./min, about-1.4° C./min to about −1.8° C./min, about −1.4° C./min to about −2° C./min, about −1.4° C./min to about-2.5° C./min, about −1.6° C./min to about −1.8° C./min, about −1.6° C./min to about −2° C./min, about-1.6° C./min to about −2.5° C./min, about −1.8° C./min to about −2° C./min, about −1.8° C./min to about-2.5° C./min, or about −2° C./min to about −2.5° C./min. In some embodiments, the sub-freeze rate is between about −0.1° C./min, about −0.2° C./min, about −0.4° C./min, about −0.6° C./min, about-0.8° C./min, about −1° C./min, about −1.2° C./min, about −1.4° C./min, about −1.6° C./min, about-1.8° C./min, about −2° C./min, or about −2.5° C./min. In some embodiments, the sub-freeze rate is between at least about −0.1° C./min, about −0.2° C./min, about −0.4° C./min, about −0.6° C./min, about-0.8° C./min, about −1° C./min, about −1.2° C./min, about −1.4° C./min, about −1.6° C./min, about-1.8° C./min, or about −2° C./min. In some embodiments, the sub-freeze rate is between at most about-0.2° C./min, about −0.4° C./min, about −0.6° C./min, about −0.8° C./min, about −1° C./min, about-1.2° C./min, about −1.4° C./min, about −1.6° C./min, about −1.8° C./min, about −2° C./min, or about-2.5° C./min. In some embodiments, the sub-freeze rate can be −1.36° C./min. In some embodiments, the sub-freeze rate comprises a range of −1.13° C./min to −1.62° C./min.

In some embodiments, wherein the supra-freezing rate, sub-freezing rate, and nucleation temperature for the given biological sample is not known, the cyrobanking methods described herein further comprise determining the supra-freezing rate, sub-freezing rate, and nucleation temperature for the biological sample. In some embodiments, the supra-freezing rate, sub-freezing rate, and nucleation temperature are derived from a freezing curve for the biological sample. In some embodiments, the freezing curve is modelled using a computer. In some embodiments, the freezing curve is determined empirically following the procedures and methods described herein.

In some embodiments, the post-thaw viability rate of the cells is at least about 60% to about 95%. In some embodiments, the post-thaw viability rate of the cells is at least about 60% to about 70%, about 60% to about 80%, about 60% to about 90%, about 60% to about 95%, about 70% to about 80%, about 70% to about 90%, about 70% to about 95%, about 80% to about 90%, about 80% to about 95%, or about 90% to about 95%. In some embodiments, the post-thaw viability rate of the cells is at least about 60%, about 70%, about 80%, about 90%, or about 95%. In some embodiments, the post-thaw viability rate of the cells is at least at least about 60%, about 70%, about 80%, or about 90%. In some embodiments, the post-thaw viability rate of the cells is at least at most about 70%, about 80%, about 90%, or about 95%. In some embodiments, the post-thaw viability rate of the cells is at least 60%. In some embodiments, the post-thaw viability rate of the cells is at least 70%. In some embodiments, the post-thaw viability rate of the cells is at least 80%. In some embodiments, the post-thaw viability rate of the cells is at least 90%.

In some embodiments, (c) occurs in one or more freezers. In some embodiments, the first container and the second container are disposed in a first freezer of the one or more freezers. In some embodiments, the first container is contained in a first freezer of the one or more freezers and the second container is contained in a second freezer of the one or more freezers. In some embodiments, the one or more freezers comprise a static freezer. In some embodiments, the first freezer, the second freezer, or both is a static freezer. method of any one of the preceding claims, wherein the one or more freezers comprise a controlled-rate freezer. In some embodiments, the first freezer, the second freezer, or both is a controlled-rate freezer. In some embodiments, the one or more freezers are set at about −70° C. to −90° C. In some embodiments, the one or more freezers are set at −80° C. In some embodiments, the one or more freezers are set at −86° C. In some cases, the one or more freezers can be set at a range of temperature from about −100° C. to about −60° C. In some cases, the freezer can be set at a range of temperature from about −60° C. to about −65° C., about −60° C. to about −70° C., about −60° C. to about −75° C., about −60° C. to about −80° C., about −60° C. to about −82° C., about −60° C. to about −84° C., about −60° C. to about −86° C., about −60° C. to about-88° C., about −60° C. to about −90° C., about −60° C. to about −95° C., about −60° C. to about −100° C., about −65° C. to about −70° C., about −65° C. to about −75° C., about −65° C. to about −80° C., about −65° C. to about −82° C., about −65° C. to about −84° C., about −65° C. to about −86° C., about −65° C. to about-88° C., about −65° C. to about −90° C., about −65° C. to about −95° C., about −65° C. to about −100° C., about −70° C. to about −75° C., about −70° C. to about −80° C., about −70° C. to about −82° C., about −70° C. to about −84° C., about −70° C. to about −86° C., about −70° C. to about −88° C., about −70° C. to about-90° C., about −70° C. to about −95° C., about −70° C. to about −100° C., about −75° C. to about −80° C., about −75° C. to about −82° C., about −75° C. to about −84° C., about −75° C. to about −86° C., about −75° C. to about −88° C., about −75° C. to about −90° C., about −75° C. to about −95° C., about −75° C. to about-100° C., about −80° C. to about −82° C., about −80° C. to about −84° C., about −80° C. to about −86° C., about −80° C. to about −88° C., about −80° C. to about −90° C., about −80° C. to about −95° C., about −80° C. to about −100° C., about −82° C. to about −84° C., about −82° C. to about −86° C., about −82° C. to about-88° C., about −82° C. to about −90° C., about −82° C. to about −95° C., about −82° C. to about −100° C., about −84° C. to about −86° C., about −84° C. to about −88° C., about −84° C. to about −90° C., about −84° C. to about −95° C., about −84° C. to about −100° C., about −86° C. to about −88° C., about −86° C. to about-90° C., about −86° C. to about −95° C., about −86° C. to about −100° C., about −88° C. to about −90° C., about −88° C. to about −95° C., about −88° C. to about −100° C., about −90° C. to about −95° C., about-90° C. to about −100° C., or about −95° C. to about −100° C. In some cases, the freezer can be set at a range of temperature from about −60° C., about −65° C., about −70° C., about −75° C., about −80° C., about −82° C., about −84° C., about −86° C., about −88° C., about −90° C., about −95° C., or about −100° C. In some cases, the freezer can be set at a range of temperature from at least about −60° C., about-65° C., about −70° C., about −75° C., about −80° C., about −82° C., about −84° C., about −86° C., about-88° C., about −90° C., or about −95° C. In some cases, the freezer can be set at a range of temperature from at most about −65° C., about −70° C., about −75° C., about −80° C., about −82° C., about −84° C., about −86° C., about −88° C., about −90° C., about −95° C., or about −100° C.

In some embodiments, the second volume is placed in an insulating container (e.g. a cooling box). In some embodiments, the method further comprises arranging the first volume inside the static freezer such that the first volume does not contact a wall of the one or more freezers. In some embodiments, the biological sample comprising cells or a derivative thereof, in the first volume and the biological sample comprising cells or a derivative thereof, in the second volume experience a same cooling rate. In some embodiments, the cells are stem cells or immune cells. In some embodiments, the stem cells comprise hematopoietic stem cells (HSC), mesenchymal stem cells (MSC), or both. In some embodiments, the biological sample comprises one or more organs, blood, or both. In some embodiments, the immune cells comprise T cells. In some embodiments, the blood is cord blood or peripheral blood. In some embodiments, the HSCs comprise CD34+ cells. It is contemplated that the containers can be in many forms. For instance, the biological sample or derivative thereof can be contained in bags of 1 ml to 5 ml volume or vials of 0.1 to 15 ml volumes. In a preferred embodiment, the samples with less than 15 ml of biological sample are stored in an insulating container (e.g. a cooling box) within a freezer.

Described herein, in some embodiments, is a method for cryopreserving bone marrow or bone marrow cells. In some embodiments, the method utilizes the systems described herein. In some embodiments, the method comprises processing bone to obtain bone marrow or derivative thereof to obtain bone marrow cells. In some cases, the bone marrow cells can be any cells that can be isolated from bone marrow. In some embodiments, the bone marrow cells can be hematopoietic stem cells. In some embodiments, the bone marrow cells can be mesenchymal stem cells. In some embodiments, the bone marrow or bone marrow cells to be cryopreserved at a freeze rate comprising at least −0.1° C./min, −0.2° C./min, −0.5° C./min, −1° C./min, −1.5° C./min, −2° C./min, −2.5° C./min, −3° C./min, −3.5° C./min, −4° C./min, −4.5° C./min, −5° C./min, −5.5° C./min, −6° C./min, −7° C./min, −7.5° C./min, −8° C./min, −8.5° C./min, −9° C./min, −9.5° C./min, −10° C./min, −11° C./min, −12° C./min, −13° C./min, −14° C./min, −15° C./min, −20° C./min, or higher rate.

In some embodiments, the freeze rate comprises the temperature decrease as measured by directly contacting the bone marrow or bone marrow cells with a thermometer. In some embodiments, the freeze rate comprises the temperature decrease as measured in the microenvironment or environment immediately adjacent the bone marrow or bone marrow cells. In some embodiments, the freeze rate comprises the temperature decrease as measured in the freezing apparatus (e.g. freezing bag, cryobag, cryotube, cryotank, freezing cassette, freezer, or vessel holding liquid nitrogen).

In some embodiments, the method of cryopreserving the bone marrow or bone marrow cells described herein increases the yield of the bone marrow cells after thawing compared to bone marrow cells that are not cryopreserved by the freezer rate described herein. In some instances, the yield of the bone marrow cells cryopreserved by the freezer rate described herein is increased by at least about 10%, 20%, 30%, 40%, 50%, 60%, 70%, 80%, 90%, 100%, 2 folds, 3 folds, 4 folds, 5 folds, 10 folds, 20 folds, 50 folds, or more compared to yield of bone marrow cells not cryopreserved by the freezer rate described herein. In some embodiments, the method of cryopreserving the bone marrow or bone marrow cells described herein increases the viability of the bone marrow cells after thawing compared to bone marrow cells that are not cryopreserved by the freezer rate described herein. In some instances, the viability of the bone marrow cells cryopreserved by the freezer rate described herein is increased by at least about 10%, 20%, 30%, 40%, 50%, 60%, 70%, 80%, 90%, 100%, 2 folds, 3 folds, 4 folds, 5 folds, 10 folds, 20 folds, 50 folds, or more compared to viability of bone marrow cells not cryopreserved by the freezer rate described herein. In some embodiments, the method of cryopreserving the bone marrow or bone marrow cells described herein increases the number of CD34+ bone marrow cells after thawing compared to the number of CD34+ bone marrow cells that are not cryopreserved by the freezer rate described herein. In some instances, the number of CD34+ the bone marrow cells cryopreserved by the freezer rate described herein is increased by at least about 10%, 20%, 30%, 40%, 50%, 60%, 70%, 80%, 90%, 100%, 2 folds, 3 folds, 4 folds, 5 folds, 10 folds, 20 folds, 50 folds, or more compared to the number of CD34+ the bone marrow cells not cryopreserved by the freezer rate described herein. In some embodiments, the method of cryopreserving the bone marrow or bone marrow cells described herein increases the number of CD45+ bone marrow cells after thawing compared to the number of CD45+ bone marrow cells that are not cryopreserved by the freezer rate described herein. In some instances, the number of CD45+ the bone marrow cells cryopreserved by the freezer rate described herein is increased by at least about 10%, 20%, 30%, 40%, 50%, 60%, 70%, 80%, 90%, 100%, 2 folds, 3 folds, 4 folds, 5 folds, 10 folds, 20 folds, 50 folds, or more compared to the number of CD45+ the bone marrow cells not cryopreserved by the freezer rate described herein.

In some embodiments, after thawing the samples frozen utilizing the schemes described herein (e.g. Example 5), the samples contain an increased amount of viable CD34+ cells as compared to known cryopreservation protocols. In some embodiments, the percentage of viable CD34+ cells in the thawed sample is at least about 70% to about 95%. In some embodiments, the percentage of viable CD34+ cells in the thawed sample is at least about 70% to about 75%, about 70% to about 80%, about 70% to about 85%, about 70% to about 90%, about 70% to about 95%, about 75% to about 80%, about 75% to about 85%, about 75% to about 90%, about 75% to about 95%, about 80% to about 85%, about 80% to about 90%, about 80% to about 95%, about 85% to about 90%, about 85% to about 95%, or about 90% to about 95%. In some embodiments, the percentage of viable CD34+ cells in the thawed sample is at least about 70%, about 75%, about 80%, about 85%, about 90%, or about 95%. In some embodiments, the percentage of viable CD34+ cells in the thawed sample is at least at least about 70%, about 75%, about 80%, about 85%, or about 90%. In some embodiments, the percentage of viable CD34+ cells in the thawed sample is at least at most about 75%, about 80%, about 85%, about 90%, or about 95%.

### Kits

In certain embodiments, described herein is a kit for preparing an allogenic composition. In some embodiments, the allogenic composition comprises an enhanced sub-population of lymphocytes as described herein. In some embodiments, the sub-population of lymphocytes comprises γδ T cells, CD8+ naïve T cells, CD4+ naïve T cells, or Natural Killer (NK) cells. In some embodiments, the kit comprises a cryopreserving solvent as described herein. In some embodiments, the allogenic composition is used to prevent or treat an auto immune disease in a subject in need thereof. In some embodiments, the kit comprises instructions for the use thereof.

In some embodiments, the cryopreserving solvent comprises an active cryopreserving moiety. In some embodiments, the active cryopreserving moiety comprises a polar aprotic compound or a zwitterionic compound. In some embodiments, the active cryopreserving moiety comprises a polar aprotic compound. In some embodiments, said active cryopreserving moiety comprises a zwitterionic compound. In some embodiments, the active cryopreserving moiety comprises dimethyl sulfoxide (DMSO); 1, 2 propane diol; propylene glycol; ethylene glycol; glycerol; foramamide; ethanediol or butane 2, 3 diol.

In some embodiments, the kit comprises a biological sample a described herein. In some embodiments, the kit comprises the buffers and tools used in cryopreservation of the biological sample as described herein. In some embodiments, the kit comprises a freeze media comprising a rinse media and a cryopreservation solution. In some embodiments, the kit comprises at least one cryopreservation bag. In some embodiments, the kit comprises a syringe.

### Definitions

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to +10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The section headings used herein are for organizational purposes only and are not to be construed as limiting the subject matter described.

The following example is included for illustrative purposes only and are not intended to limit the scope of the invention.

### Example 1: Cryopreservation of Bone Marrow in 2.5%, 5% and 10% DMSO

Bone marrow prior to DMSO addition (designated “QC1”) was washed and resuspended at a concentration of 8×10⁶ cells/ml in Rinse Media. Cells were aliquoted and mixed with equal volumes of DMSO solutions to yield final concentrations of either 2.5, 5 or 10% DMSO in Rinse Media. A 2.5 ml volume of each preparation was aliquoted in triplicate to 5 ml cryovials. After equilibrating for 5 minutes, the cryovials were placed in a CryoMed Controlled Rate Freezer and frozen using the following program:
Step 1. −1° C./minute to −86° C.
Step 2. −20° C./minute to −150° C.
Step 3. Hold at −150° C.

The vials were transferred to LN2 after 5 minutes at −150° C. The following day, cryovials containing cryopreserved cells were thawed in a 37° C. water bath and transferred to a biological safety cabinet after sanitizing with 70% alcohol. The cells were transferred to a centrifuge tube and pelleted. After removing the supernatant, the cells were resuspended in 12.5 ml of Selection Media. The tubes were centrifuged again and the cell pellet was resuspended in final volume of 25 ml of Selection Media. Total cell counts were obtained using a Sysmex Hematology Analyzer.

Flow cytometry was performed using non-cryopreserved Hematopoietic cells and bone marrow QC1 sample for comparison. Panel design for flow cytometry with the following panel of conjugated antibodies (see Table 1). Each tube was stained with the viability dye 7-AAD after antibody staining.

TABLE 1
Panel design for flow cytometry

| Group A: natural killer cells | | | | Group C: naïve and | |
|---|---|---|---|---|---|
| and CD19+ B cells | | Group B: γ δ T cells. | | memory CD4 T cells. | |
| 5 ml | APC anti- | 2 ml | PE anti- | 5 ml | APC-Cy7 anti- |
| | human CD3 | | human CD3 | | human CD4 |
| 5 ml | FITC anti- | 5 ml | APC-Cy7 | 5 ml | PE anti-human |
| | human CD16 | | anti-human | | CD45RA |
| | | | CD4 | | |
| 5 ml | PE-Cy7 anti- | 5 ml | PE-Cy7 anti- | 5 ml | APC anti- |
| | human CD19 | | human CD8 | | human |
| | | | | | CD45RO |
| 20 ml | PE anti-human | 5 ml | APC anti- | 5 ml | PE-Cy7 anti- |
| | CD56 | | human gd | | human CCR7 |
| | | | TCR | | |
| Group D: naïve and memory | | Group E: hematopoietic | | | |
| CD8 T cells. | | stem/progenitor cells. | | | |
| 5 ml | FITC anti- | 5 ml | APC anti- | | |
| | human CD8 | | human CD34 | | |
| 5 ml | PE anti-human | 5 ml | FITC anti- | | |
| | CD45RA | | human CD45 | | |
| 5 ml | APC anti- | | | | |
| | human | | | | |
| | CD45RO | | | | |
| 5 ml | PE-Cy7 anti- | | | | |
| | human CCR7 | | | | |

CFU assays were also performed.

### Comparison of Fresh Versus Cryopreserved Hematopoietic Cells and Bone Marrow.

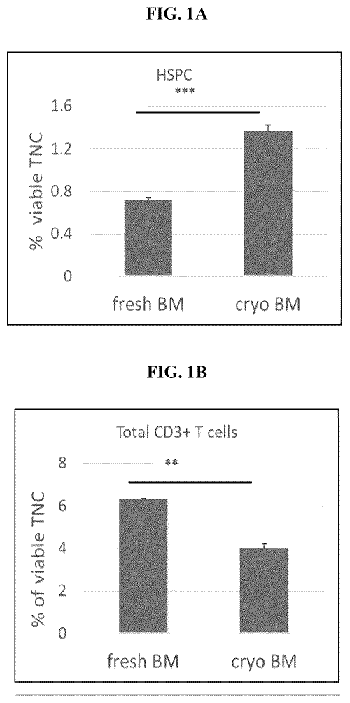

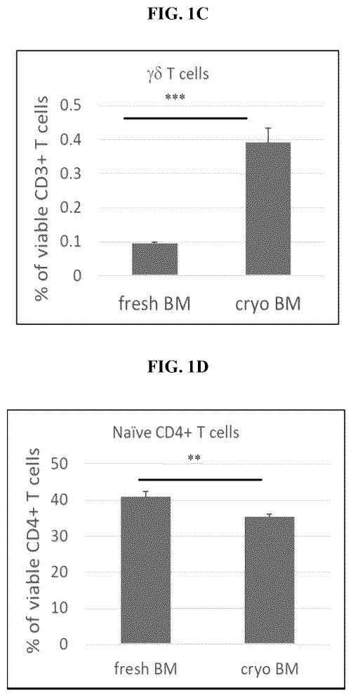

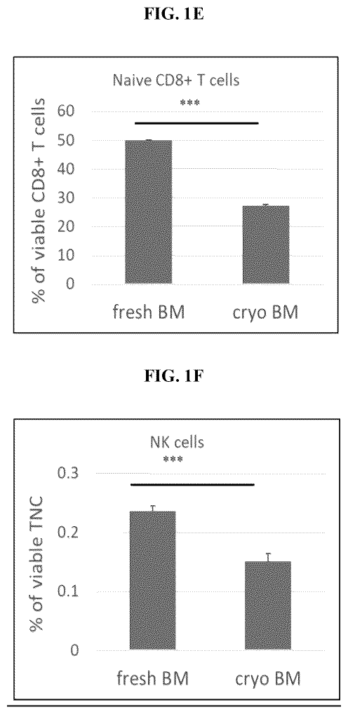

The percentage of CD45^{LO}CD34+ HSPC and lymphocytes subsets was compared before and after cryopreserving Hematopoietic cells and bone marrow in 5% DMSO (FIG. 1A to FIG. 1F). The percentage of HSPC within the total viable cell population (labeled “% of viable TNC”) increased with cryopreservation (FIG. 1A); whereas, the total population of T cells (CD3+) decreased (FIG. 1B). However, there was a significant increase in the percentage of GVL-promoting gd T cell with cryopreservation compared to fresh preparations of Hematopoietic cells and bone marrow (FIG. 1C). The percentage of potentially GVHD-promoting CD4+/CD8+CDRA+CCL7+ naïve T cells significantly decreased with cryopreservation (FIG. 1D and FIG. 1E). The percentage of NK cells also decreased following cryopreservation (FIG. 1F)

### Comparison Between Different DMSO Concentrations Used to Cryopreserve Hematopoietic Cells and Bone Marrow

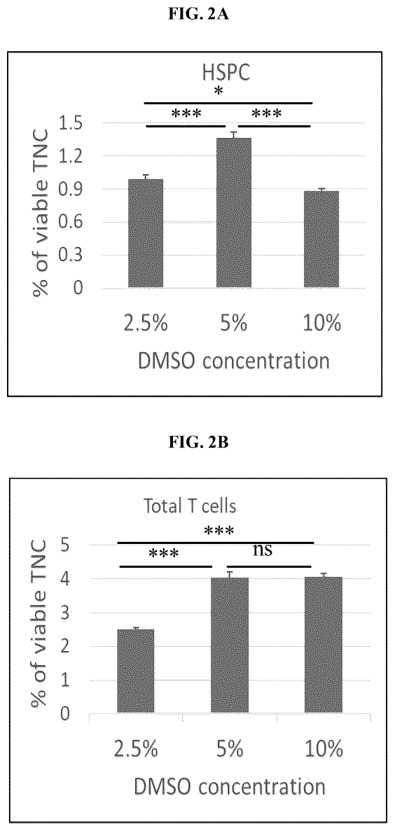

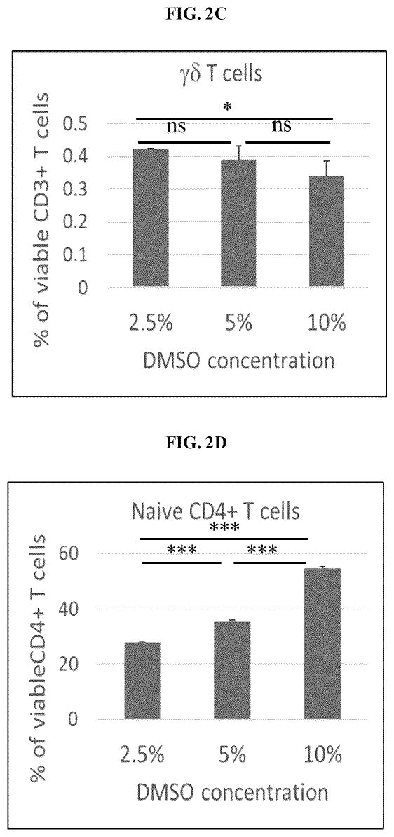

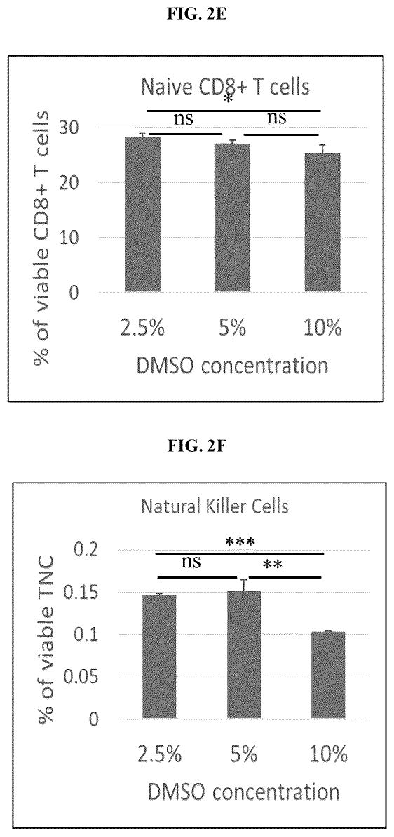

Hematopoietic cells and bone marrow (QC1) cryopreserved in either 2.5, 5 or 10% DMSO was thawed and analyzed by flow cytometry (FIG. 2A to FIG. 2F) and CFU assay (Table 2). The percentage of viable CD45^{LO}CD34+ HSPC in thawed products was highest with cryopreservation in 5% DMSO (FIG. 2A). Total CD3+ T cells was reduced with cryopreservation in 2.5% DMSO compared to either 5 or 10% (FIG. 2B). The percentage of CD3+CD4-CD8-gd TCR+gd T cells was lowest with cryopreservation in 10% DMSO compared to 2.5 and 5% (FIG. 2C). The percentage of GVHD-inducing CD4+CDRA+CCR7+ naïve T cells increased with increasing DMSO concentrations (FIG. 2D). There was a trend in decreasing naïve CD8+ T cells when cryopreserved with higher DMSO concentrations, which reached significance between 2.5 and 10% DMSO (FIG. 2E). Natural Killer cells (CD3-CD19-CD56+) cells were highest in either 2.5 or 5% DMSO (FIG. 2F).

### CFU Assay.

Fresh and as well as previously cryopreserved hematopoietic cells and bone marrow preparations were analyzed by CFU assay. The average scores for each colony type and total CFU from duplicate wells are presented in Table 2. Given the limited sample size, statistical analysis could not be performed. Qualitatively, though, it appears that 10% DMSO significantly reduces Hematopoietic cells and bone marrow CFU potential compared to 2.5 or 5% DMSO.

TABLE 2
CFU assay results for Hematopoietic cells and bone marrow before cryopreservation
(fresh) or after thawing cells cryopreserved in 2.5, 5 or 10% DMSO.

| | CFU-E | BFU-E | CFU-GM | CFU-GEMM | total |
|---|---|---|---|---|---|
| fresh bone marrow | 9.5 | 21.5 | 37.5 | 1 | 69.5 |
| cryopreserved in 2.5% DMSO | 7.5 | 11.5 | 7.5 | 0.5 | 27 |
| cryopreserved in 5% DMSO | 6 | 11.5 | 9 | 0.5 | 27 |
| cryopreserved in 10% DMSO | 3.5 | 4 | 0 | 0 | 7.5 |

CFU-E, Colony forming unit erythroid; BFU-E, burst forming unit- erythroid; CFU-GM, colony forming unit- granulocyte/monocyte; CFU-GEMM, colony forming unit-granulocyte/erythroid/monocyte/megakaryocyte.

### CONCLUSIONS

Cryopreservation in 5% DMSO reduced the overall number of T cells and specifically the naïve T cell subsets which are implicated in inducing GVHD. Conversely the percentage of HSPC for engraftment and gd T cells, which facilitate engraftment and reduce GVHD and may be involved in GVT, both increased with cryopreservation. When compared to 2.5 and 10% DMSO in the cryopreservation medium, 5% DMSO enhanced the percentage of HSPC without a loss of GVL-promoting NK cells or reduced numbers of T cells. The percentage of CD4 naïve T cells was also lowest with the lower DMSO concentrations. Both gd T cells and CD8+ naïve T cells were relatively insensitive to DMSO concentration.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for preparing an allogenic composition from a biological sample, wherein said allogenic composition comprises an enhanced sub-population of lymphocytes, the method comprising cryopreserving said biological sample in a cryopreserving solvent comprising less than 2 molar concentration of an active cryopreserving moiety.

2. The method of claim 1, wherein the method further comprises generating a second subpopulation of lymphocytes, generating a population of natural killer (NK) cells, or any combination thereof from said biological sample.

3. The method of claim 1, wherein the method further comprises depleting a third sub-population of lymphocytes from said biological sample.

4. The method of claim 1, wherein said sub-population of lymphocytes comprises γδ T cells, CD8+ naive T cells, CD4+ naive T cells, or any combination thereof.

5. The method of claim 1, wherein said biological sample is derived from a donor subject.

6. The method of claim 1, wherein said biological sample comprises blood, bone marrow, peripheral blood, cord blood, or any combination thereof.

7. The method of claim 1, wherein said active cryopreserving moiety comprises an amphiphilic compound, a polar aprotic compound, a zwitterionic compound, or any combination thereof.

8. The method of claim 1, wherein said active cryopreserving moiety comprises dimethyl sulfoxide (DMSO); 1, 2 propane diol; propylene glycol; ethylene glycol; glycerol; formamide; ethanediol or butane 2, 3 diol.

9. The method of claim 1, wherein said sub-population of lymphocytes comprises greater than 0.2% of said biological sample.

10. The method of claim 2, wherein said population of NK cells comprises greater than 0.1% of said biological sample.

11. A cellular composition comprising an enhanced subpopulation of lymphocytes, an enhanced second subpopulation of lymphocytes, and a depleted third subpopulation of lymphocytes, wherein said cellular composition further comprises a cryopreserving solvent comprising less than 2 molar concentration of an active cryopreserving moiety.

12. The cellular composition of claim 11, wherein said sub-population of lymphocytes comprises γδ T cells, CD8+ naive T cells, CD4+ naive T cells, or any combination thereof.

13. The cellular composition of claim 11, wherein said biological sample is derived from a donor subject.

14. The cellular composition of claim 11, wherein said biological sample comprises blood, bone marrow, peripheral blood, cord blood, or any combination thereof.

15. The cellular composition of claim 11, wherein said active cryopreserving moiety comprises an amphiphilic compound, a polar aprotic compound, a zwitterionic compound, or any combination thereof.

16. The cellular composition of claim 11, wherein said active cryopreserving moiety comprises dimethyl sulfoxide (DMSO); 1, 2 propane diol; propylene glycol; ethylene glycol; glycerol; formamide; ethanediol or butane 2, 3 diol.

17. The cellular composition of claim 11, wherein said sub-population of lymphocytes comprises greater than 0.2% of said biological sample, or wherein said third sub-population of lymphocytes comprises less than 60% of said biological sample.

18. The cellular composition of claim 11, wherein the cellular composition is allogenic.

19. A method of treating an auto-immune disease in a subject in need thereof, the method comprising:
(i) cryopreserving a biological sample in a cryopreserving solvent comprising less than 2 molar concentration of an active cryopreserving moiety to generate an allogenic composition, wherein said allogenic composition comprises an enhanced subpopulation of lymphocytes; and
(ii) administering said allogenic composition to said subject.

20. The method of claim 19, wherein said auto immune disease is graft-versus-host disease.
